(19) United States
(12) Patent Application Publication
(10) Pub. No.: US 2012/0101164 A1
(43) Pub. Date: Apr. 26, 2012

(54) ABSCISSION AND CROP STORAGE UNIT

(75) Inventors: Richard Henry Williams, Colchester (GB); Peter Roose, Ghent (BE); Johan Josef De Saegher, Ghent (BE)

(73) Assignee: TAMINCO, NAAMLOZE VENNOOTSCHAP, Ghent (BE)

(21) Appl. No.: 13/263,635

(22) PCT Filed: Apr. 7, 2010
(86) PCT No.: PCT/IB2010/001033

§ 371 Date: Jan. 11, 2012

(30) Foreign Application Priority Data
Apr. 7, 2009 (EP) 09447010.1

(51) Int. Cl. A01N 37/44; A01N 37/18; C07C 229/56

(52) U.S. Cl. 514/567; 562/458; 514/629

(57) ABSTRACT

A composition comprising a compound selected from an auxin, an auxin precursor, an auxin metabolite or a derivative of said auxin, auxin precursor or auxin metabolite or a mixture thereof and optionally acetaminophen or a derivative thereof for improving post-harvest storability of crops.

### FIELD OF THE INVENTION

The invention relates to a method and composition for improving post-harvest storability of crops by either treating plants or the harvested crop with, particularly but not exclusively, anthranilic acid, optionally in combination with acetaminophen, or an auxin in combination with acetaminophen. The shelf life of the treated crops can also be improved by this treatment.

This invention also relates to a method and composition for delaying abscission in plants by treating the plants with, particularly but not exclusively, anthranilic acid, optionally in combination with acetaminophen, or an auxin in combination with acetaminophen.

### BACKGROUND OF THE INVENTION

Obtaining the best possible yield in terms of quality and quantity is extremely important in the harvesting of crops. It is also very important that the crop can be stored until its sale or use without a reduction in its quality, and that it does not succumb to post-harvest diseases during this period. The possibility of storage of a crop for a prolonged period of time is also advantageous.

Abscission is the process by which a plant intentionally drops one or more of its parts, such as a leaf, fruit, flower or seed. A plant will abscise a part either to discard a member that is no longer necessary, such as a leaf during autumn, or a flower following fertilization, or for the purposes of reproduction. Most deciduous plants drop their leaves by abscission before winter, while evergreen plants continuously abscise their leaves. Another form of abscission is pre-harvest fruit drop, when a plant abscises fruit while still immature in order to conserve resources needed to bring the remaining fruit to maturity. If a leaf is damaged a plant may also abscise it to conserve water or photosynthetic efficiency, depending on the ‘costs’ to the plant as a whole. However, abscission of plant parts may be undesirable to the plant cultivator since, for example, pre-harvest abscission of fruits results in crop wastage.

Thus, the delay of abscission either pre-harvest or at harvest is important in maximizing yield and reducing wastage of crops and also increases the harvest period. This is especially relevant for the harvest of fruits. Prolonging the lifespan of individual plant organs such as leaves, flowers, seeds and/or fruit may also be desirable, for example to prolong petal retention in situ for flowering plants, for delaying seed loss in grasses, and reducing pod shatter in oilseed rape.

Therefore there is a strong need for effective abscission delay agents. Abscission control or reduction agents available to date may be hazardous, and may cause damage to the fruit or crop in question or an undesirable acceleration in maturity.

There is also a strong need for effective agents to prolong crop storability and improve the quality of the stored crop and delay the onset of post-harvest crop diseases, which are currently responsible for large amounts of wastage. The pesticides which have been used to-date to combat post-harvest diseases are often ineffective and pose hazards to humans and the environment. Many of these pesticides have now been withdrawn from use by regulatory agencies due to concerns over the residue on crops and over the health risks posed. In addition, an increased resistance to pesticide treatment by the pathogens responsible for post-harvest diseases has been observed.

Naphthaleneacetic acid (NAA) is a commonly used abscission control/reduction agent. However, there is a risk of acceleration in maturity and it can result in foliar injury when applied at high doses. NAA also does not display any post-harvest benefits such as improving crop storage quality, prolonging crop storability and delaying the onset of post-harvest diseases.

Thus, the need remains for additional agents to impart pre- and post-harvest benefits to crops such as delaying abscission, prolonging crop storability, improving the quality of post-harvest crops or delaying the onset of post harvest diseases and which may provide improved properties.

### SUMMARY OF THE INVENTION

The present invention relates to the novel use of a composition comprising a compound selected from an auxin, an auxin precursor, an auxin metabolite or a derivative of said auxin, auxin precursor or auxin metabolite or a mixture thereof and optionally acetaminophen or a derivative thereof for imparting post-harvest benefits to plants or crops.

The present invention also relates to the novel use of a composition comprising a compound selected from an auxin, an auxin precursor, an auxin metabolite or a derivative of said auxin, auxin precursor or auxin metabolite or a mixture thereof and optionally acetaminophen or a derivative thereof for delaying abscission in plants.

The present invention further relates to the novel use of anthranilic acid or its derivatives for imparting post-harvest benefits to plants or crops.

The present invention also relates to the novel use of anthranilic acid or its derivatives for delaying abscission in plants.

Anthranilic acid is used as an intermediate for production of dyes, pigments and saccharin. It and its esters are also used in preparing perfumes to imitate jasmine and orange, pharmaceuticals (loop diuretics such as furosemide) and UV-absorbers, as well as corrosion inhibitors for metals and mold inhibitors in soya sauce. Its usefulness for imparting post-harvest benefits to plants or stored crops is surprising.

Acetaminophen is widely used as an over-the-counter analgesic and antipyretic. It will be appreciated that its efficacy as part of a package that delays abscission in plants is surprising. It will also be appreciated that its efficacy as part of a package that imparts post-harvest benefits to plants or to stored crops is surprising.

### STATEMENTS OF THE INVENTION

The present invention is directed to the treatment of a plant with an effective amount of a composition comprising a compound selected from an auxin, an auxin precursor, an auxin metabolite or a derivative of said auxin, auxin precursor or auxin metabolite or a mixture thereof and acetaminophen or a derivative thereof for improving post-harvest storability of crops.

According to one aspect of the present invention there is provided a composition comprising a compound selected from an auxin, an auxin precursor, an auxin metabolite or a derivative of said auxin, auxin precursor or auxin metabolite or a mixture thereof and acetaminophen or a derivative thereof for improving post-harvest storability of crops.

By improving post-harvest storability of crops we mean at least one of improving the quality of the stored crop, prolonging the crop storability i.e. increasing the length of time for which the crop can be stored, delaying the onset of senescence and delaying the onset of post-harvest diseases.

The present invention also provides a composition comprising a compound selected from an auxin, an auxin precursor, an auxin metabolite or a derivative of said auxin, auxin precursor or auxin metabolite or a mixture thereof and acetaminophen or a derivative thereof wherein the auxin precursor is not AN for improving post-harvest storability of crops.

In one embodiment the auxin-related compound is based on an indolic ring. In another embodiment the auxin-related compound is based on a phenolic ring.

In one embodiment the derivative is an acid, a conjugate, a salt, an ester, or an amide of the auxin, auxin precursor, or auxin metabolite.

In one embodiment the derivative is in the form of a conjugate, e.g. conjugated to a sugar, an alcohol, an amino acid, a peptide or a protein.

In one embodiment the auxin precursor is chorismate, anthranilic acid, phosphoribosyl anthraniliate, 1-(O-carboxyphenulamino)-1-deoxyribulose-5-phosphate, indole-3-glycerol-phosphate, indole, indole-3-acetic acid, tryptophan, tryptamine, N-hydroxy tryptamine, indole-3-acetaldoxime, 1-aci-nitro-2-indolylethane, indolic glucosinate, indole-3-acetonitrile (IAN), indole-3-acetaldehyde, indole-3-lactic acid, indole-3-pyruvic acid, or indole-3-ethanol.

In a particularly preferred embodiment the auxin precursor is anthranilic acid or a derivative thereof as set out above.

The auxin-related compound may be a natural, such as is obtainable from seaweed or alage, or synthetic auxin.

In one embodiment the natural auxin is indole-3-acetic acid (IAA), 4-chloro-indole-3-acetic acid (4-Cl-IAA), phenylacetic acid (PAA), indole-3-butyric acid (IBA), indole-3-acetyl-1-O-β-D-glucose (IAAglc).

In one embodiment the conjugate of the natural auxin is IAA-Inositol, IAA-Inositol-arabinose, IAP1, an IAA-peptide, an IAA glycoprotein, an IAA-glucan, IAA-aspartate, IAA-glucose, IAA-1-O-glucose, IAA-myo-Inositol, IAA-4-O-glucose, IAA-6-O-glucose, IAA-Inositol-galactose, an IAA amide conjugate, or an IAA-amino acid conjugate.

In one embodiment the synthetic auxin is 1-naphthaleneacetic acid (NAA), 2,4-dichlorophenoxyacetic acid (2,4-D), 2-methoxy-3,6-dichlorobenzoic acid (dicamba), 4-amino-3,5,6-trichloropicolinic acid (tordon), 2,4,5-trichlorophenoxyacetic acid (2,4,5-T), 2,3,6-trichlorobenzoic acid, 4-chloro-2 methylphenoxyacetic acid (MCPA) or N,N-dimethylethylthiocarbamate.

In one embodiment the auxin metabolite is indole-3-lactic acid or indole-3-ethanol.

In one embodiment the acetaminophen derivative is a compound as set out in FIG. 3.

The present invention is also directed to the treatment of a plant with an effective amount of the compound anthranilic acid (also referred to as “AN”) or an effective salt, ester, or amide thereof including analogs of the AN and effective salts, ester and amides thereof, in order to improve post-harvest storability of crops.

Thus, according to one aspect of the present invention there is provided anthranilic acid or an analog or a derivative thereof for use for improving post-harvest storability of crops.

In one embodiment the derivative of AN or its analog is a salt, an ester, or an amide of the acid, or a conjugate of any of the foregoing.

In one embodiment the derivative compound used in the present invention is in the form of a conjugate, e.g. conjugated to a sugar, an alcohol, an amino acid, a peptide or a protein.

In one embodiment the analog of AN is a compound having the structure shown in FIG. 1.

In another embodiment there is provided a composition comprising a compound selected from an auxin, an auxin precursor, an auxin metabolite or a derivative of said auxin, auxin precursor or auxin metabolite or a mixture thereof and a further agrochemically acceptable component for improving post-harvest storability of crops, and optionally acetaminophen or a derivative thereof.

In another aspect of the present invention there is provided a composition for improving post-harvest storability of crops comprising an auxin-related compound as described above in combination with an abscission control or reduction agent.

Examples of such further abscission control or reduction agents are selected from 1-naphthaleneacetic acid (NAA), NAAm, 2,4-D, 2,4,5-T, 2,4,5-TP, amino-oxyacetic acid, aminoethoxyvinylglycine (AVG) and daminozide.

In one embodiment the further agrochemically acceptable component comprises at least one compound selected from a) glucose, hydrolysed starch, sucrose, fructose, glycerol, glyceraldehydes, erythrose, ribulose, xylulose or arabinose, monosaccharides including aldoses such as D-Ribose, D-Xylose, L-Arabinose, D-Glucose, D-Mannose and D-Galactose; ketoses such as D-Ribulose and D-Fructose; deoxyaldoses such as 2-Deoxy-D-ribose, L-Fuccose; acetylated amino sugars such as N-Acteyl-D-glucosamine and N-Acetyl-D-galactosamine; acidic monosaccharides such as D-Glucuronic acid, L-Iduronic acid and N-Acetylneuraminic acid, Sugar alcohols such as D-Sorbitol and D-Mannitol, disaccharides including maltose, lactose and sucrose, or an ester or glycoside or metabolic equivalent of such a carbohydrate; b) an organic acid of the Krebs tricarboxylic acid cycle or a metabolic precursor thereof; c) a vitamin or coenzyme, or a precursor thereof; d) a purine or pyrimidine nucleoside, nucleotide or metabolic precursor thereof; e) a naturally occurring fat or oil; or f) an amino acid.

Preferably the further agrochemically acceptable component comprises at least one compound selected from c) a vitamin or coenzyme, or a precursor thereof; d) a purine or pyrimidine nucleoside, nucleotide or metabolic precursor thereof; or f) an amino acid.

In one embodiment the AN-related compound or the composition of the present invention is for improving post-harvest storability of crops.

In another aspect of the present invention there is provided a method of improving post-harvest storability of crops comprising applying an effective amount of the AN-related compound or the composition of the present invention to a plant either pre-harvest or post-harvest, or to the plant's locus, or to a harvested crop.

Thus the present invention relates to a method for applying to plants or harvested crops an effective amount of an AN-related compound alone or in combination with simultaneous or sequential applications of acetaminophen or a derivative thereof and/or another suitable ingredient for improving post-harvest storability of crops and the crop quality after a shelf-life period.

The present invention also relates to a method for applying to plants or harvested crops an effective amount of an AN-related compound alone or in combination with simultaneous or sequential applications of acetaminophen or a derivative thereof and/or another suitable ingredient for improving the crop quality after a shelf-life period.

The present invention also relates to a method for applying to plants or harvested crops an effective amount of an auxin-related compound in combination with simultaneous or sequential applications of acetaminophen or a derivative thereof and/or another suitable ingredient for improving post-harvest storability of crops and the crop quality after a shelf-life period.

The present invention also relates to a method for applying to plants or harvested crops an effective amount of an auxin-related compound in combination with simultaneous or sequential applications of acetaminophen or a derivative thereof and/or another suitable ingredient for improving the crop quality after a shelf-life period.

In one embodiment the AN-related compound or the composition of the present invention is for use as a post-harvest crop storage quality improver.

In one embodiment premature germination or sprouting in harvested crops is delayed.

Examples of crops in which premature germination or sprouting may be delayed include potato, cereal crops and grain and pod-bearing crops.

In one embodiment turgor is maintained.

Examples of crops in which turgor is maintained include lettuce and celery.

In a further embodiment the AN-related compound or the composition of the present invention is for use in prolonging crop storability.

By prolonging crop storability we mean increasing the length of time for which a crop can be stored. In particular, this means increasing the “shelf-life” or “vase-life”. Shelf-life is the length of time that food, for example fruit or vegetables, can be kept before it is considered unsuitable for sale or consumption. Vase-life is the length of time that cut flowers maintain their decorative appearance. An increased vase-life means that cut flowers can be displayed, or stored before sale or display, for a longer period of time.

In a still further embodiment the AN-related compound or the composition of the present invention is for use in delaying post-harvest diseases and preventing pests.

Non-limiting examples of post-harvest diseases include viral, bacterial or fungal diseases.

Non-limiting examples of such plants include trees, shrubs, vines, vegetables or ornamental crops.

The crop can be a fruit, vegetable, or agricultural or horticultural crop, including ornamentals.

Non-limiting examples of such fruits include pome fruit, stone fruit, citrus fruit or kiwi fruit. In one embodiment the plant is an apple, pear, plum, cherry, apricot, peach, citrus or nectarine tree, a grapevine or kiwi plants.

In one embodiment the AN-related compound or the composition of the present invention is applied to the plant pre-harvest, at harvest or after harvest.

In another embodiment the AN-related compound or the composition of the present invention is applied to the crop post-harvest.

In a further embodiment the AN-related compound or the composition of the present invention is applied to the crop post-harvest by means of a spray, dips or drenches.

The present invention is further directed to the treatment of a plant with an effective amount of a composition comprising a compound selected from an auxin, an auxin precursor, an auxin metabolite or a derivative of said auxin, auxin precursor or auxin metabolite or a mixture thereof and acetaminophen or an analog or derivative thereof in order to delay abscission in that plant.

The present invention is also directed to the treatment of a plant with an effective amount of the compound anthranilic acid (also referred to as “AN”) or an effective salt, ester, or amide thereof including analogs of the AN and effective salts, ester and amides thereof, in order to delay abscission in that plant.

By analog we include a compound that has a similar structure, i.e same of similar active moiety, and similar chemical properties, e.g. with AN is capable of delaying abscission in plants.

According to another aspect of the present invention there is provided a composition comprising a compound selected from an auxin, an auxin precursor, an auxin metabolite or a derivative of said auxin, auxin precursor or auxin metabolite or a mixture thereof and acetaminophen or an analog or derivative thereof for use as an abscission delay agent.

The present invention also provides a composition comprising a compound selected from an auxin, an auxin precursor, an auxin metabolite or a derivative of said auxin, auxin precursor or auxin metabolite or a mixture thereof and acetaminophen or an analog or derivative thereof wherein the auxin precursor is not AN for use as an abscission delay agent.

For ease of reference we will also refer to auxin, an auxin precursor, an auxin metabolite or a derivative of said auxin, auxin precursor or auxin metabolite or derivative of said auxin, auxin precursor or auxin metabolite as an “auxin-related compound”.

By “agrochemically acceptable component” we include components that are tolerated by a plant, and ideally which are beneficial to a plant.

In one embodiment the auxin-related compound is based on an indolic ring. In another embodiment the auxin-related compound is based on a phenolic ring.

In one embodiment the derivative is an acid, a conjugate, a salt, an ester, or an amide of the auxin, auxin precursor, or auxin metabolite.

In one embodiment the derivative is in the form of a conjugate, e.g. conjugated to a sugar, an alcohol, an amino acid, a peptide or a protein.

In one embodiment the auxin precursor is chorismate, anthranilic acid, phosphoribosyl anthraniliate, 1-(O-carboxyphenulamino)-1-deoxyribulose-5-phosphate, indole-3-glycerol-phosphate, indole, indole-3-acetic acid, tryptophan, tryptamine, N-hydroxy tryptamine, indole-3-acetaldoxime, 1-aci-nitro-2-indolylethane, indolic glucosinate, indole-3-acetonitrile (IAN), indole-3-acetaldehyde, indole-3-lactic acid, indole-3-pyruvic acid, or indole-3-ethanol.

In a particularly preferred embodiment the auxin precursor is anthranilic acid or a derivative thereof as set out above.

The auxin-related compound may be a natural, such as is obtainable from seaweed or algae, or synthetic auxin.

In one embodiment the natural auxin is indole-3-acetic acid (IAA), 4-chloro-indole-3-acetic acid (4-Cl-IAA), phenylacetic acid (PAA), indole-3-butyric acid (IBA), indole-3-acetyl-1-O-β-D-glucose (IAAglc).

In one embodiment the conjugate of the natural auxin is IAA-Inositol, IAA-Inositol-arabinose, IAP1, an IAA-peptide, an IAA glycoprotein, an IAA-glucan, IAA-aspartate, IAA-glucose, IAA-1-O-glucose, IAA-myo-Inositol, IAA-4-O-glucose, IAA-6-O-glucose, IAA-Inositol-galactose, an IAA amide conjugate, or an IAA-amino acid conjugate.

In one embodiment the synthetic auxin is 1-naphthaleneacetic acid (NAA), 2,4-dichlorophenoxyacetic acid (2,4-D), 2-methoxy-3,6-dichlorobenzoic acid (dicamba), 4-amino-3,5,6-trichloropicolinic acid (tordon), 2,4,5-trichlorophenoxyacetic acid (2,4,5-T), 2,3,6-trichlorobenzoic acid, 4-chloro-2 methylphenoxyacetic acid (MCPA) or N,N-dimethylethylthiocarbamate.

In one embodiment the auxin metabolite is indole-3-lactic acid or indole-3-ethanol.

In one embodiment the acetaminophen derivative is a compound as set out in FIG. 3.

According to one aspect of the present invention there is provided anthranilic acid or an analog or a derivative thereof for use as an abscission delay agent. In other words the present invention is a composition for delaying abscission in plants comprising, or consisting essentially of, or consisting of one or more of AN or an analog or a derivative thereof.

In one embodiment the derivative of AN or its analog is a salt, an ester, or an amide of the acid, or a conjugate of any of the foregoing.

In one embodiment the derivative compound used in the present invention is in the form of a conjugate, e.g. conjugated to a sugar, an alcohol, an amino acid, a peptide or a protein.

In one embodiment the analog of AN is a compound having the structure shown in FIG. 1.

For ease of reference we will refer to all of the above mentioned AN, analogs and derivatives thereof as “AN-related compounds”.

In another embodiment there is provided a composition comprising a compound selected from an auxin, an auxin precursor, an auxin metabolite or a derivative of said auxin, auxin precursor or auxin metabolite or a mixture thereof and a further agrochemically acceptable component for use as an abscission delay agent, and optionally acetaminophen or a derivative thereof.

In another aspect of the present invention there is provided a composition for use as an abscission delay agent comprising an auxin-related compound as described above in combination with a further abscission control or reduction agent.

Examples of such further abscission control or reduction agents include 1-naphthaleneacetic acid (NAA), NAAm, 2,4-D, 2,4,5-T, 2,4,5-TP, amino-oxyacetic acid, aminoethoxyvinylglycine (AVG) and daminozide.

In one embodiment the further agrochemically acceptable component comprises at least one compound selected from a) glucose, hydrolysed starch, sucrose, fructose, glycerol, glyceraldehydes, erythrose, ribulose, xylulose or arabinose, monosaccharides including aldoses such as D-ribose, D-xylose, L-arabinose, D-glucose, D-mannose and D-galactose; ketoses such as D-ribulose and D-fructose; deoxyaldoses such as 2-deoxy-D-ribose, L-fucose; acetylated amino sugars such as N-acteyl-D-glucosamine and N-acetyl-D-galactosamine; acidic monosaccharides such as D-glucuronic acid, L-iduronic acid and N-acetylneuraminic acid, sugar alcohols such as D-sorbitol and D-mannitol, disaccharides including maltose, lactose and sucrose, or an ester or glycoside or metabolic equivalent of such a carbohydrate; b) an organic acid of the Krebs tricarboxylic acid cycle or a metabolic precursor thereof; c) a vitamin or coenzyme, or a precursor thereof; d) a purine or pyrimidine nucleoside, nucleotide or metabolic precursor thereof; e) a naturally occurring fat or oil; or f) an amino acid.

Preferably the further agrochemically acceptable component comprises at least one compound selected from c) a vitamin or coenzyme, or a precursor thereof; d) a purine or pyrimidine nucleoside, nucleotide or metabolic precursor thereof; or f) an amino acid.

In one embodiment the AN-related compound or the composition of the present invention is for use as an abscission delay agent to delay fruit drop prior to or at harvest.

In another embodiment the AN-related compound or the composition of the present invention is for use as an abscission delay agent to delay seed loss e.g. in cereals and grasses.

In a further embodiment the AN-related compound or the composition of the present invention is for use as an abscission delay agent to reduce pod shatter e.g. in oilseed rape.

In a still further embodiment the AN-related compound or the composition of the present invention is for use as an abscission delay agent to prolong the lifespan of individual plant organs such as leaves, flowers, seeds or fruit e.g. to prolong petal retention in situ for flowering plants.

In another aspect of the present invention there is provided a method of delaying abscission in plants comprising applying a chemically effective amount of the AN-related compound or the composition of the present invention to a plant, or its locus.

Thus the present invention relates to a method for applying to plants an effective amount of an AN-related compound alone or in combination with simultaneous or sequential applications of acetaminophen or a derivative thereof and/or another suitable ingredient to delay abscission.

The present invention also relates to a method for applying to plants an effective amount of an auxin-related compound in combination with simultaneous or sequential applications of acetaminophen or a derivative thereof and/or another suitable ingredient to delay abscission.

Non-limiting examples of such plants include trees, shrubs or crops.

By “crop” we include cultivated plants and agricultural or horticultural produce. This may include the desired proceeds/produce of a harvested plant, which may be either whole-plant (total biomass) or the portion of the plant which is useful, such as fruit, vegetables, ornamental plants, flowers, seeds etc.

The plant part for which abscission or senescence is delayed can be leaves, fruit, vegetables, ornamentals, flowers or seeds.

Non-limiting examples of such fruits include pome fruit, stone fruit, citrus fruit or kiwi fruit. In one embodiment the plant is an apple, pear, plum, cherry, apricot, peach or nectarine tree, grapevine, citrus or kiwi.

As described herein the components of the compositions of the present invention may be applied at the same or different times. Thus, we provide a kit of the components wherein at least one component is in a separate container.

We have found that the combinations disclosed herein may give rise to a synergistic effect in relation to the applications disclosed herein, such as improving post-harvest storability, abscission control etc.

### ADVANTAGES

We have found that an AN-related compound, and particularly AN, when applied alone or in mixture with other agrochemically acceptable compounds is (amongst other benefits in plants or harvested crops) effective in delaying abscission and senescence in plants, wherever this is deemed useful.

We have found that an auxin-related compound, and more particularly an AN-related compound, and even more particularly AN, when applied with acetaminophen as a combination or in mixture with other agrochemically acceptable compounds is (amongst other benefits in plants or harvested crops) effective in delaying abscission and senescence in plants, wherever this is deemed useful.

We have also found that an auxin-related compound, and more particularly an AN-related compound, and even more particularly AN, when applied with acetaminophen in mixture with an additive such as at least one compound selected from a) glucose, hydrolysed starch, sucrose, fructose, glycerol, glyceraldehydes, erythrose, ribulose, xylulose or arabinose, monosaccharides including aldoses such as D-Ribose, D-Xylose, L-Arabinose, D-Glucose, D-Mannose and D-Galactose; ketoses such as D-Ribulose and D-Fructose; deoxyaldoses such as 2-Deoxy-D-ribose, L-Fuccose; acetylated amino sugars such as N-Acteyl-D-glucosamine and N-Acetyl-D-galactosamine; acidic monosaccharides such as D-Glucuronic acid, L-Iduronic acid and N-Acetylneuraminic acid, Sugar alcohols such as D-Sorbitol and D-Mannitol, disaccharides including maltose, lactose and sucrose, or an ester or glycoside or metabolic equivalent of such a carbohydrate; b) an organic acid of the Krebs tricarboxylic acid cycle or a metabolic precursor thereof; c) a vitamin or coenzyme, or a precursor thereof; d) a purine or pyrimidine nucleoside, nucleotide or metabolic precursor thereof; e) a naturally occurring fat or oil; or f) an amino acid is (amongst other benefits in plants or harvested crops) an effective abscission or senescence delay agent, wherever this is deemed useful.

We have found that the above compounds and compositions act as an effective abscission delay agent when added to a range of species. Such compounds or compositions would be helpful either when used on their own, or in combination with abscission control or reduction agents on the market, e.g. in order to improve their safety, efficacy or economic attractiveness.

We have found that the present invention can provide less phytotoxicity, and/or no acceleration of maturity in comparison to commercial standard abscission control or reduction agents.

We have found that an AN-related compound, and particularly AN, when applied alone or in mixture with other agrochemically acceptable compounds is (amongst other benefits in plants or harvested crops) effective in imparting post-harvest benefits to crops, wherever this is deemed useful.

We have found that an auxin-related compound, and more particularly an AN-related compound, and even more particularly AN, when applied with acetaminophen as a combination or in mixture with other agrochemically acceptable compounds is (amongst other benefits in plants or harvested crops) effective in imparting post-harvest benefits to crops, wherever this is deemed useful.

We have also found that an auxin-related compound, and more particularly an AN-related compound, and even more particularly AN, when applied with acetaminophen in mixture with an additive such as at least one compound selected from a) glucose, hydrolysed starch, sucrose, fructose, glycerol, glyceraldehydes, erythrose, ribulose, xylulose or arabinose, monosaccharides including aldoses such as D-Ribose, D-Xylose, L-Arabinose, D-Glucose, D-Mannose and D-Galactose; ketoses such as D-Ribulose and D-Fructose; deoxyaldoses such as 2-Deoxy-D-ribose, L-Fuccose; acetylated amino sugars such as N-Acteyl-D-glucosamine and N-Acetyl-D-galactosamine; acidic monosaccharides such as D-Glucuronic acid, L-Iduronic acid and N-Acetylneuraminic acid, Sugar alcohols such as D-Sorbitol and D-Mannitol, disaccharides including maltose, lactose and sucrose, or an ester or glycoside or metabolic equivalent of such a carbohydrate; b) an organic acid of the Krebs tricarboxylic acid cycle or a metabolic precursor thereof; c) a vitamin or coenzyme, or a precursor thereof; d) a purine or pyrimidine nucleoside, nucleotide or metabolic precursor thereof; e) a naturally occurring fat or oil; or f) an amino acid is (amongst other benefits in plants or harvested crops) an effective agent for imparting post-harvest benefits to crops, wherever this is deemed useful.

We have found that the above compounds and compositions act as an effective agent for imparting post-harvest benefits to crops when added to a range of species. Such compounds or compositions would be helpful either when used on their own, or in combination with other abscission control or reduction agents on the market, e.g. in order to improve their safety, efficacy or economic attractiveness.

### BRIEF DESCRIPTION OF THE FIGURES

FIG. 1 shows structures of examples of analogs of anthranilic acid.

FIG. 2 shows structures of examples of naturally occurring auxins and conjugates.

FIG. 3 shows structures of examples of derivatives of acetaminophen.

FIG. 4 shows an overview of the reactions leading from chorismate to IAA and tryptophan.

FIG. 5 shows the structure of some synthetic auxins.

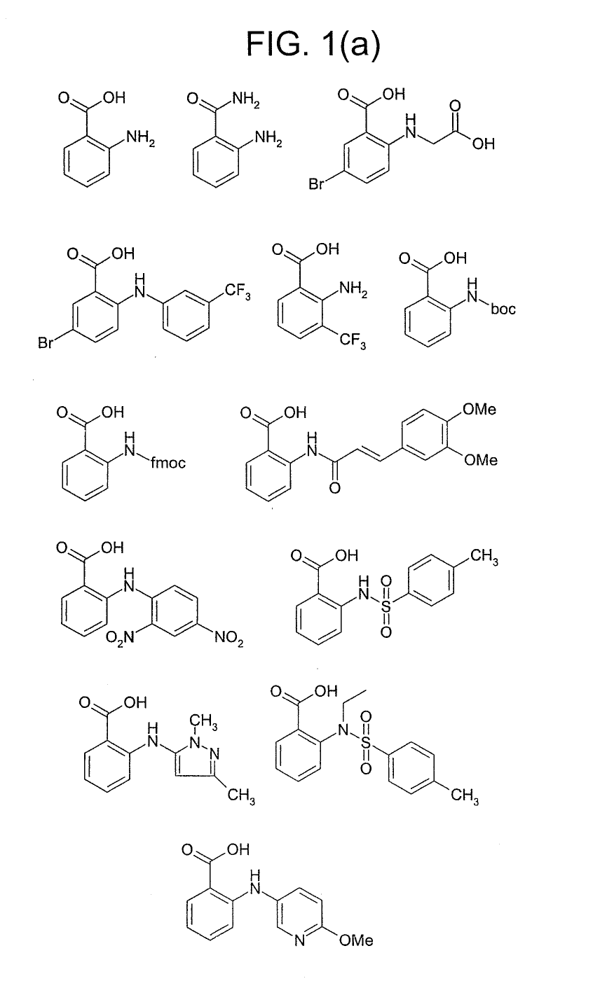

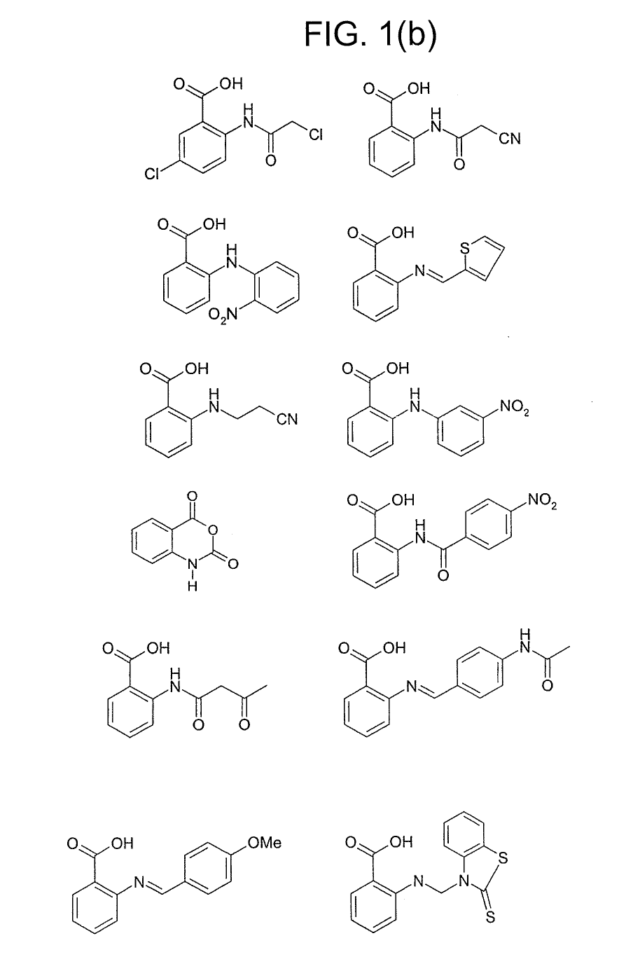

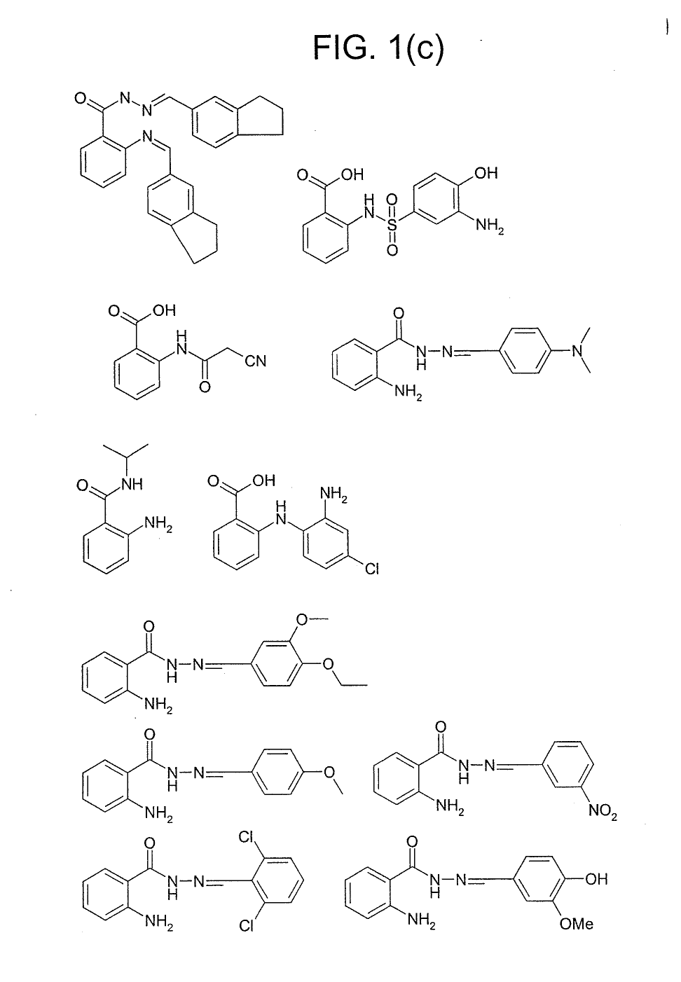

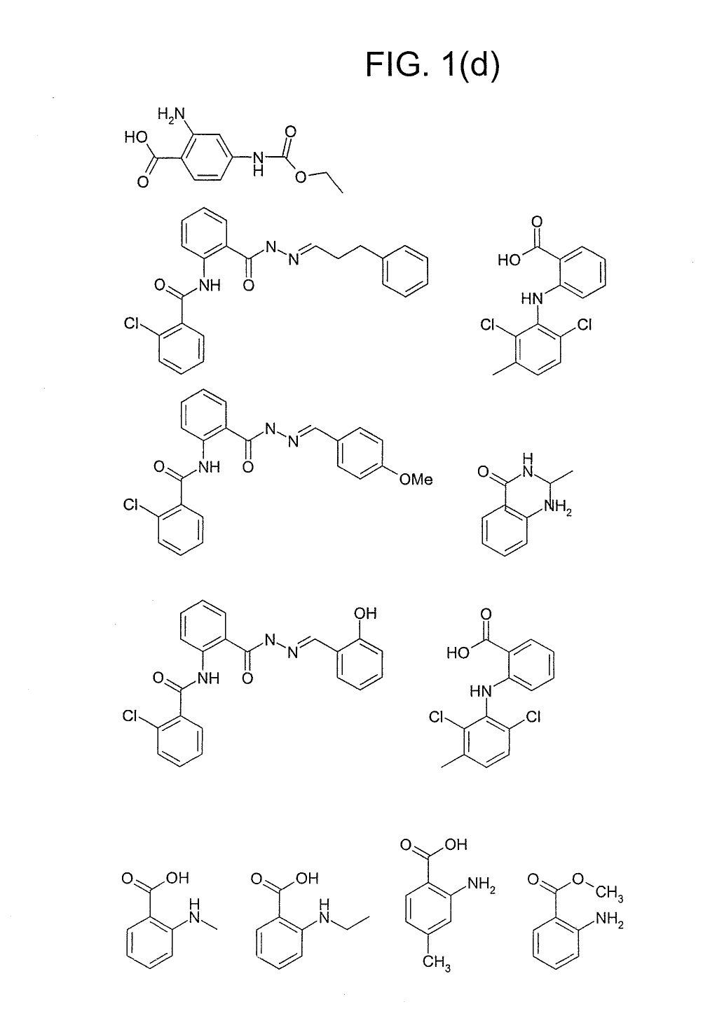

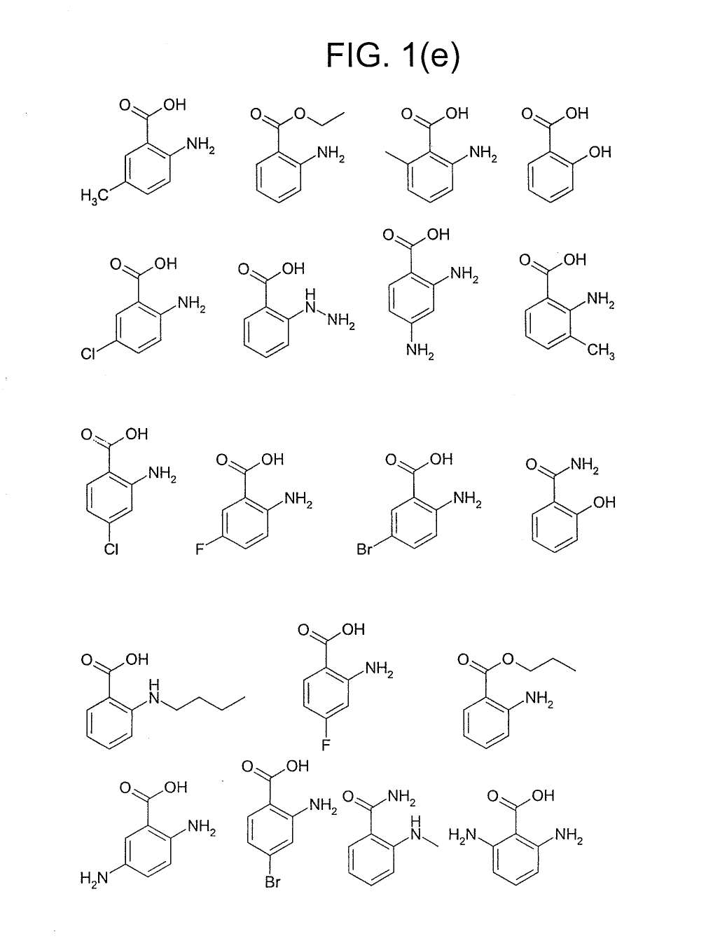

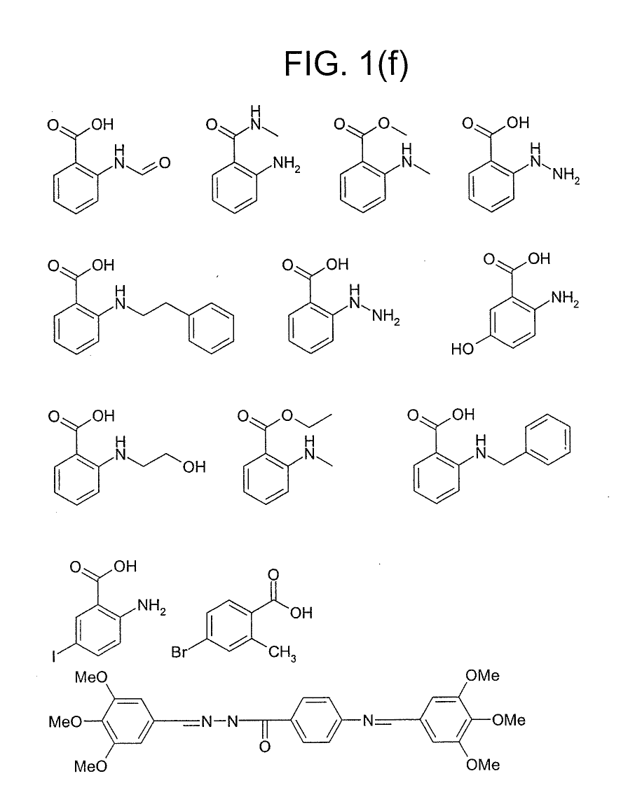

### DETAILED DESCRIPTION OF THE INVENTION

Various preferred features and embodiments of the present invention will now be described by way of non-limiting example.

The invention provides a process and a compound or a composition for delaying abscission and senescence in plants. The process of the invention includes applying an effective amount of a compound or composition to the blossoms and/or fruit of a fruit bearing plant, to the flowers of a flowering plant, to vegetables, to the seeds of cereals and grasses, and to the pods of pod-bearing and other crops.

The invention also provides a process and a compound or a composition for imparting post-harvest benefits to crops. The post-harvest benefits include improving crop storability, i.e. prolonging crop storage times and improving the quality of stored post-harvest crops, delaying of the onset of post-harvest diseases, delaying premature germination, prolonging shelf-life or vase-life, maintaining turgor and suppressing sprouting. The process of the invention includes applying an effective amount of a compound or composition to the blossoms and/or fruit of a fruit bearing plant, to the flowers of a flowering plant, to the seeds of cereals and grasses, and to the pods of pod-bearing and other crops pre-harvest. The process of the invention also includes applying an effective amount of a compound or composition to whole plants with roots intact or harvested crops post-harvest.

By “effective amount” we include an amount of the compound or composition of the present invention which is sufficient to achieve the desired “abscission delay response”, the desired “senescence delay response”, or the desired “crop storability response”, as appropriate. In general by “abscission delay response” we mean delaying abscission in plants e.g. delaying fruit drop prior to harvest compared to a control. In general by “senescence delay response” we mean delaying senescence in plants i.e. delaying aging. In general by “crop storability response” we mean prolonging crop storage times and improving the quality of stored post-harvest crops, and delaying the onset of post-harvest diseases.

The present invention relates to the use of anthranilic acid (AN):

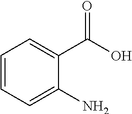

AN, also known as anthraniliate, has the CAS number 118-92-3.

We have described useful derivatives of AN above. Preferably such derivatives are water soluble. Representative salts include inorganic salts such as ammonium, lithium, sodium, potassium, magnesium and calcium salts and organic amine salts such as the triethanolamine, dimethylethanolamine and ethanolamine salts.

The present invention involves the use of auxins.

Auxins are a class of plant growth hormones. An auxin is an organic substance that promotes cell elongation growth when applied in low concentrations to plant tissue segments in a bioassay. The most studied member of the auxin family is indole-3-acetic acid (IAA). In addition to IAA, there are several other naturally occurring auxins that have been described to date: IAA, IBA, PAA and 4-Cl-IAA. Naturally occurring auxins are found in plants as the free acid and in conjugated forms.

An auxin has been defined as a compound that gives rise to curvature in the grass coleoptile curvature (or growth) test. Such an assay is described by Fritz Went in 1926 and 1928. In this bioassay coleoptile tips of grass seedlings are placed on an agar plate containing the substance to be assayed. If an auxin response is present then the coleoptile bends in darkness and the angle of curvature can be measured. Went's results indicated that the curvatures of stems were proportional to the amount of growth substance in the agar. This test is also called the avena curvature test. Other functional tests which can be employed to determine auxin activity include the ability to cause rooting in stem cuttings and the ability to promote cell division in tissue or cell culture.

A review of auxins, their synthesis and metabolism can be found in e.g. Normanly, Slovin and Cohen in “Plant Hormones, Biosynthesis, Signal Transduction and Action!”, Ed Peter J. Davies, [2004] Chapter “B1. Auxin Biosynthesis and Metabolism” pages 36-62.

In addition to indolic auxins, various phenolic auxins have auxin activity.

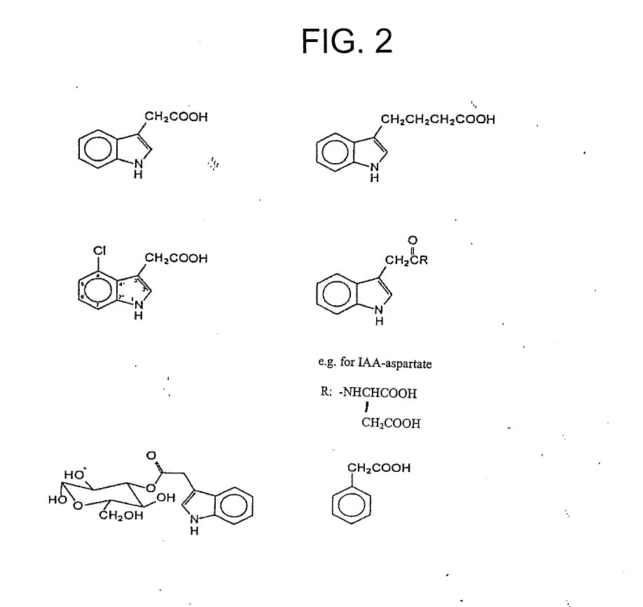

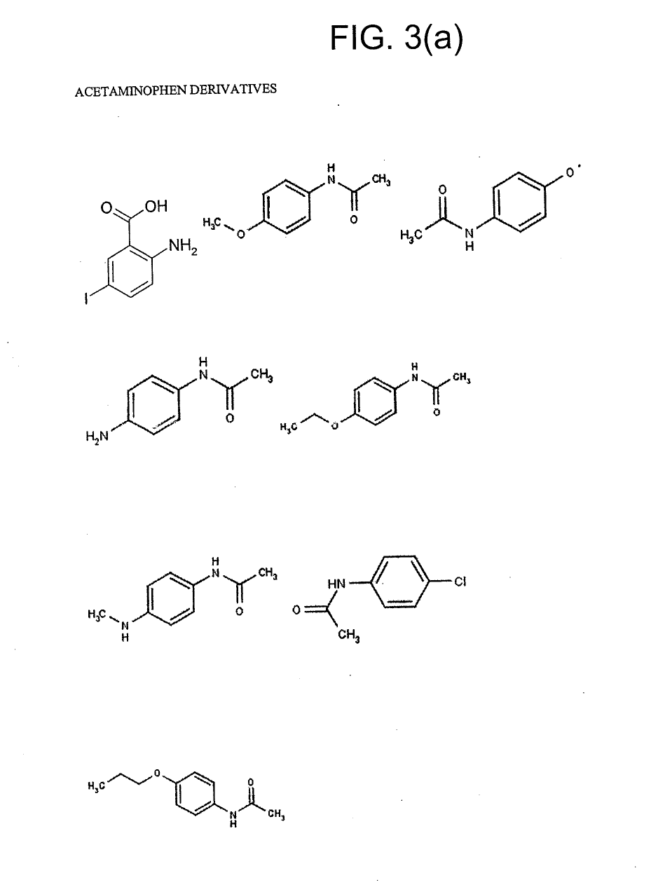

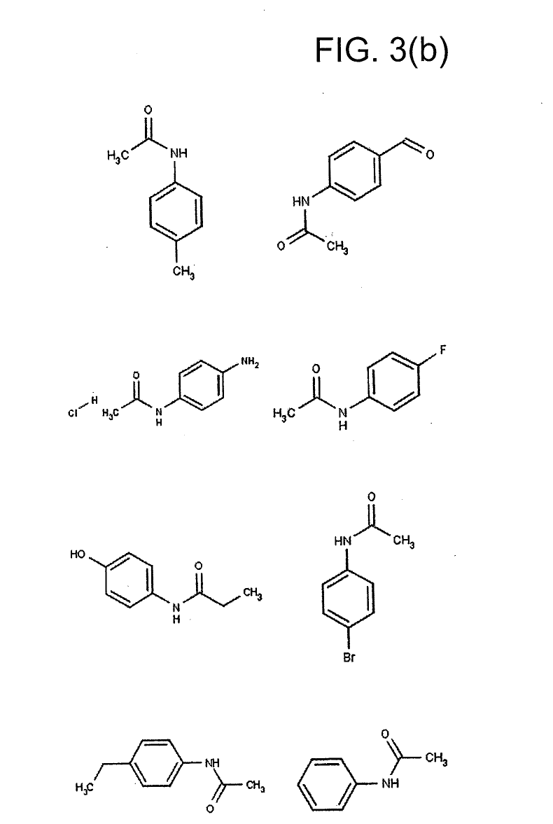

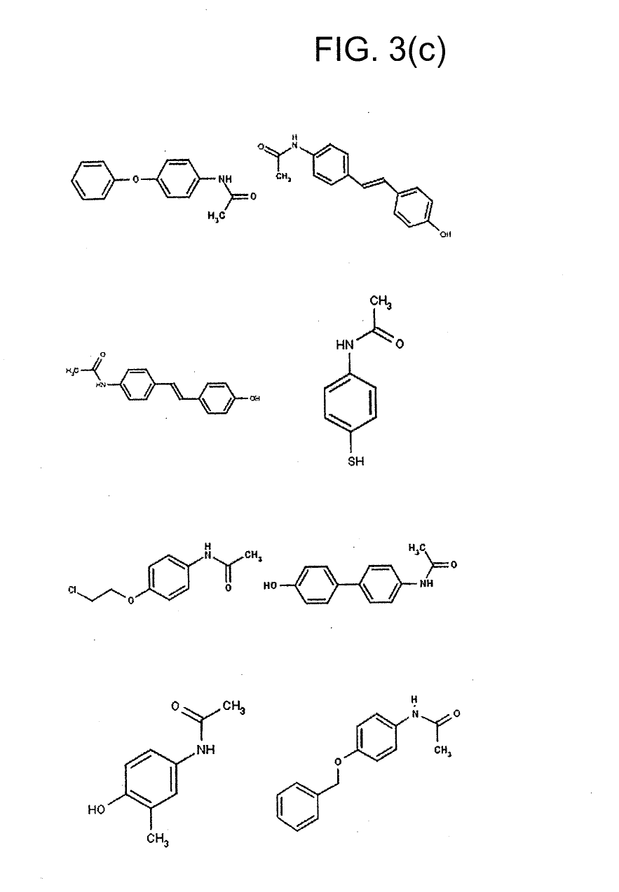

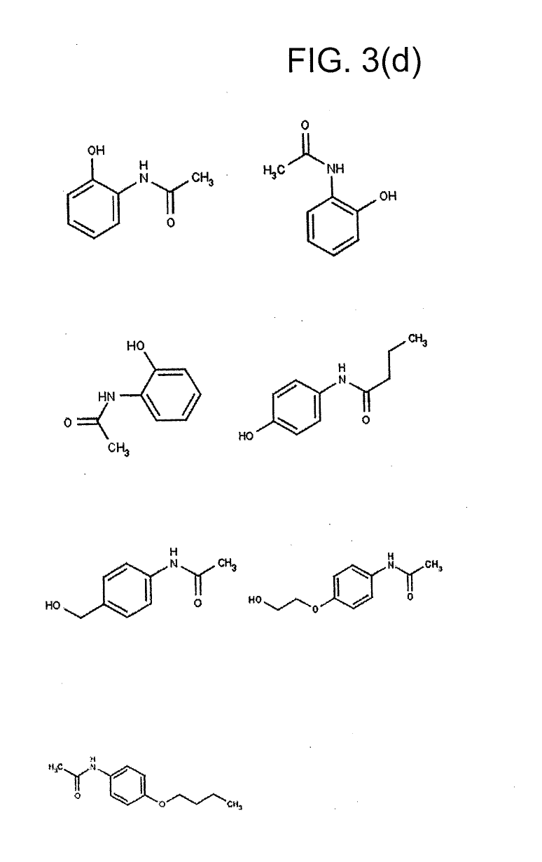

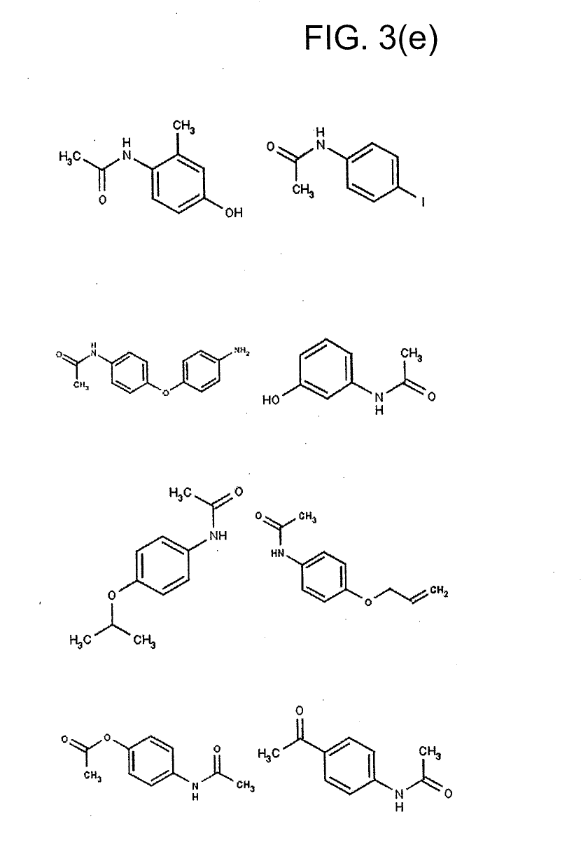

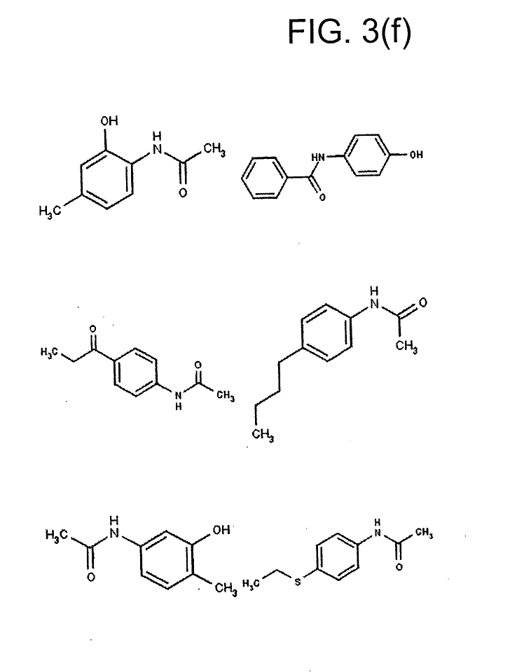

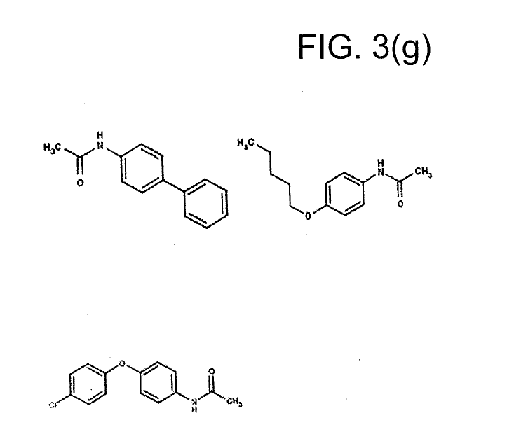

Some examples of naturally occurring auxins and some examples of the lower molecular weight conjugates which may be used in the present invention are shown in FIG. 2.

The present invention may also make use of conjugates. It is believed that plants use conjugates for storage purposes and/or to regulate the amount of free auxin available in the plant. IAA is primarily conjugated to the amino acid aspartate.

Related low molecular weight conjugates, such as IAA-Inos, IAA-Inos-arabinose and conjugates with other amino acids, and higher molecular weight conjugates, such as the IAA protein IAP1, IAA-peptides, IAA glycoprotein and IAA-glucans, have also been isolated from plants.

IAA and its precursors undergo metabolic conversions to indole-3-lactic acid, indole-3-ethanol and IBA. IBA has been found to occur naturally in plants; although some references refer to it as a synthetic auxin. Some commentators refer to it as an auxin per se and others as a precursor to IAA.

One general class of conjugated forms consists of those linked through carbon-oxygen-carbon bridges. These compounds have been referred to generically as “ester-linked”, although some 1-O sugar conjugates such as 1-O-IAA-Gluc are actually linked by acyl alkyl acetal bonds. Typical ester-linked moieties include 6-O-IAGluc, IAA-Inos, IAA-glycoproteins, IAA-glucans and simple methyl and ethyl esters. The other type of conjugates present in plants are linked through carbon-nitrogen-carbon amide bonds (referred to as “amide-linked”), as in the IAA-amino acid and protein and peptide conjugates (see FIG. 2).

Biochemical pathways that result in IAA production within a plant tissue include: (A) de novo synthesis, whether from tryptophan [referred to as Trp-dependent (Trp-D) IAA synthesis], or from indolic precursors of Trp [referred to as Trp-independent (Trp-I) IAA synthesis, since these pathways bypass Trp]; (B) hydrolysis of both amide- and ester-linked IAA conjugates; (C) transport from one site in the plant to another site; and (D) conversion of IBA to IAA. IAA turnover mechanisms include: (E) oxidative catabolism; (F) conjugate synthesis; (G) transport away from a given site; and (H) conversion of IAA to IBA. The present invention makes use of such precursors and metabolites along this pathway. The present invention does not make use of inactive metabolites, such as arise from catabolism of the auxin.

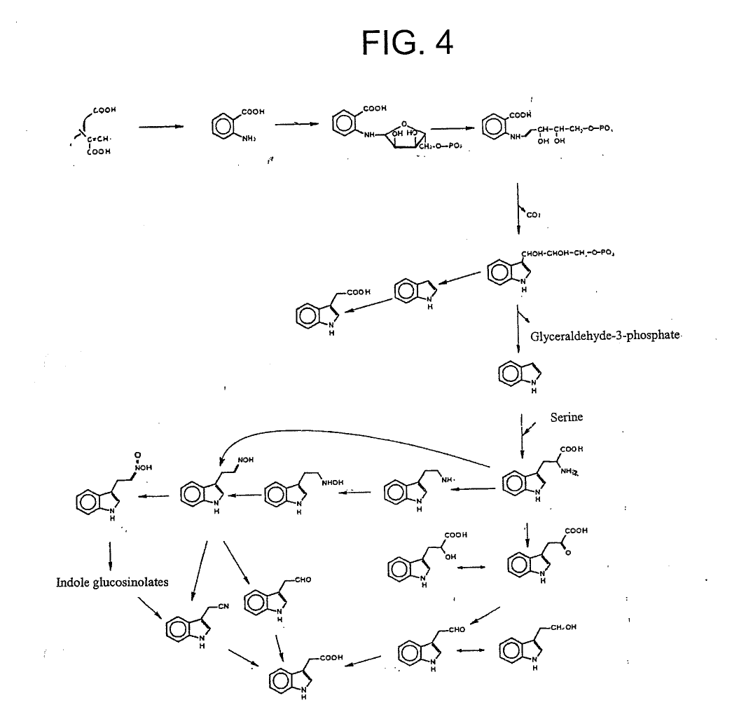

Normally the present invention makes use of the tryptophan-dependent pathway. A summary of the reactions leading from chorismate—the first committed step of indolic metabolism—to IAA and tryptophan is shown in FIG. 4.

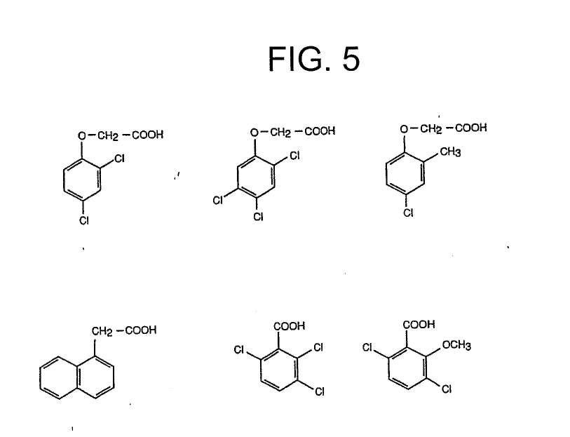

The present invention also encompasses the use of synthetic auxins. Some examples of synthetic auxins are shown in FIG. 5.

A comparison of the compounds that possess auxin activity reveals that at neutral pH they all have a strong negative charge on the carboxyl group of the side chain that is separated from a weaker positive charge on the ring structure by a distance of about 0.5 nm. It has been proposed that an indole is not essential for activity, but that it can be an aromatic or fused aromatic ring of a similar size. A model has been proposed as being a planar aromatic ring-binding platform, a carboxylic acid-binding site and a hydrophobic transition region that separates the two binding sites.

The present invention involves the use of acetaminophen in some embodiments.

Acetaminophen has the IUPAC name, N-(4-hydroxypheyl)acetamide and is commonly referred to as paracetamol. It has the CAS number 103-90-2.

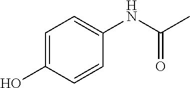

As described above, derivatives of acetaminophen are also useful in the present invention.

The compounds or compositions of the present invention can be used in combination with other components, as appropriate.

In one preferred embodiment one such component may be an additive as defined as belonging to one or more of the following classes (a) to (f); although two or more such additives in the same or different classes may be used:
(a) glucose, hydrolysed starch, sucrose, fructose, glycerol, glyceraldehyde, erythrose, xylulose or arabinose, monosaccharides including aldoses such as D-Ribose, D-Xylose, L-Arabinose, D-Glucose, D-Mannose and D-Galactose; ketoses such as D-Ribulose and D-Fructose; deoxyaldoses such as 2-Deoxy-D-ribose, L-Fuccose; acetylated amino sugars such as N-Acteyl-D-glucosamine and N-Acetyl-D-galactosamine; acidic monosaccharides such as D-Glucuronic acid, L-Iduronic acid and N-Acetylneuraminic acid, Sugar alcohols such as D-Sorbitol and D-Mannitol, disaccharides including maltose, lactose and sucrose, or an ester or glycoside or metabolic equivalent of such a carbohydrate, which will normally be applied at 10 to 10,000 g/ha (grams per hectare). Without wishing to be bound by any theory the component may function as
(1) A source for the production of high energy bonds as in adenosine trisephosphate (ATP) production,
(2) For the formation of reduced nicotinamide adenine dinucleotide (NADH) and reduced nicotamide adenine dinucleotide phosphate (NADPH) and
(3) As precursors of amino acids and nucleotides;
(b) an organic acid of the Krebs Tricarboxylic Acid Cycle or a metabolic precursor thereof, (including citric, succinic, malic, pyruvic, acetic and fumaric acids), which will normally be applied at similar rates to and used for similar functions as the carbohydrate source;
(c) a vitamin or coenzyme, e.g. thiamine, riboflavin, pyridozine, pyridoxamine, pyridoxal, nicotinamide, folic acid, or a precursor thereof including nicotinic acid, which will normally be applied at 0.01 to 500 g/ha to stimulate metabolic processes dependent on enzymatic action;
(d) a purine or pyrimidine nucleoside, nucleotide or a metabolic precursor thereof, e.g. adenine, adenosine, thymine, thymidine, cytosine, guanine, guanosine, hypoxanthine, uracil, uridine or inosine, which will normally be applied at 1 to 500 g/ha to act as structural precursors for nucleic acid synthesis;
(e) a naturally occurring fat or oil including olive, soya, coconut and corn oils, which can be degraded by living organisms to fatty acids and which will normally be applied at 10 to 10,000 g/ha;
(1) an amino acid of a type that occurs naturally in plant proteins, e.g. glycine, alanine, valine, leucine, isoleucine, serine, threonine, cysteine, methionine, aspartic acid, glutamic, acid, glutamine, asparagine, lysine, hyroxylysine, arginine, histidine, phenylalanine, tyrosine, tryptophan, proline or hydroxyproline, which will normally be applied at 1 to 500 g/ha to act as structural units for newly formed proteins or by their degradation to function in a similar manner to fatty acids and carbohydrates.

Other ingredients such as adjuvants may be added to the solution comprising the compounds or compositions of the present invention. The adjuvants can facilitate spreading and efficacy, and improve the adhesion properties of the composition, and generally include oils, antifoaming agents and surfactants. Such components which are useful in the present invention include, but are not limited to: terpene, Brij family (polyoxyethylene fatty alcohol ether) from Uniqema (Castle, Del.); surfactant in Tween family (Polyoxyethylene sorbitan esters) from Uniqema (Castle, Del.); Silwet family (Organosilicone) from Union Carbide (Lisle, Ill.); Triton family (Octylphenol ethoxylate) from The Dow Chemical Company (Midland, Mich.); Tomadol family (ethoxylated linear alcohol) from Tomah3 Products, Inc. (Milton, Wis.); Myrj family (Polyoxyethylene (POE) fatty acid esters) from Uniqema (Castle, Del.); Span family (Sorbitan ester) from Uniqema (Castle, Del.); and Trylox family (Ethoxylated Sorbitol and Ethoxylated Sorbitol Esters) from Cognis Corporation (Cincinnati, Ohio) as well as commercial surfactant Latron B-1956 (77.0% modified phthalic/glycerol alkyl resin and 23.0% Butyl alcohol) from Rohm & Haas (Philadelphia, Pa.); Caspil (Blend of Polyether-polymethylsiloxanecopolymer and nonionic surfactant) from Aquatrols (Paulsboro, N.J.); Agral 90 (Nonyl phenol ethoxylate) from Norac Concept, Inc. (Orleans, Ontario, Canada); Kinetic (99.00% Proprietary blend of polyalkyleneoxide modified polydimethylsiloxane and nonionic surfactants) from Setre Chemical Company (Memphis, Tenn.); and Regulaid (90.6% 2-butoxyethanol, poloxalene, monopropylene glycol) from KALO, Inc. (Overland Park, Kans.).

When the final solution comprising the compounds or compositions of the present invention and optionally other ingredients is to be applied to plants which, because of their hairy or waxy surface, may be difficult to wet, it may be particularly advantageous to include such other additives, commonly known in the agrochemical industry, such as surfactants, wetting agents, spreaders and stickers. (Examples of wetting agents include silicone surfactants, nonionic surfactants such as alkyl ethoxylates, anionic surfactants such as phosphate ester salts and amphoteric or cationic surfactants such as fatty acid amido alkyl betaines).

The compounds or compositions of the invention may be the sole active ingredient of the composition or they may be admixed with one or more additional active ingredients such as nematicides, insecticides, synergists, herbicides, fungicides, fertilisers or plant growth regulators where appropriate.

The compounds or compositions of the present invention can also be used in combination with other abscission control or reduction agent such as, for example, 1-naphthaleneacetic acid (NAA), NAAm, 2,4-D, 2,4,5-T, 2,4,5-TP, amino-oxyacetic acid, aminoethoxyvinylglycine (AVG) and daminozide.

Abscission delay is the delay of the abscission process prior to or at harvest through the addition of a compound that delays the abscission process in plants. Abscission of plant parts such as fruits, vegetables, flowers, petals, leaves, seeds and pods may be delayed. The abscission delay compounds and compositions of the present invention can have dramatic effects on crops such as fruits, vegetables, cereals and grasses, pod-bearing and other crops: (1) increased yield and crop quality; (2) increased time window in which harvesting can be performed; (3) increasing the shelf-life or vase-life or crops linked to a delay in natural senescence; (4) delaying premature germination or sprouting of seeds or produce; (5) maintenance of quality benefits, such as hagberg falling number etc in quality wheat, and other quality parameters in other crops; (6) obviating or reducing the need for application of pesticides pre-harvest.

Abscission control or reduction agents are commonly applied before abscission becomes a problem, preferably shortly before abscission becomes a problem. The exact timing of the addition of an abscission control or reduction agent depends on material chosen, cultivar, climatological condition, local conditions and the plant part for which a delay in abscission is required, e.g. flowers, petals, leaves, fruit, vegetables, seeds and pods. Many factors affect the abscission of plant parts, including the presence or absence of nearby fruit and young leaves, daylength, water status, soil salinity, soil nutrient levels, the level of toxins in the soil, light intensity, pests and diseases, mechanical damage, temperature extremes, drought and the activity of applied compounds such as plant growth regulators.

For example, abscission control or reduction agents are generally applied by fruit growers each season. The degree to which abscission is delayed depends on a number of factors: variety and strain, tree condition, fruit set, proximity to pollinizets, plant part subject to abscission, weather, the chemical, and application method.

Imparting post-harvest benefits to plants or post-harvest crops is improving the storage ability of crops through the addition of a compound that improves the quality of stored crops post-harvest. The compounds and compositions of the present invention which impart these post-harvest benefits can have a number of dramatic effects, such as: (1) prolonging crop storability; (2) improving the quality of post-harvest crops; and (3) delaying the onset of natural senescence and of post harvest diseases; (4) improved shelf-life or vase-life of the produce; (5) delaying premature germination or sprouting or seeds or produce; (6) maintaining turgor; (7) obviating or reducing the requirement for use of pesticide on crops pre- or post-harvest; (8) suppressing sprouting. For example, agents used to impart post-harvest benefits to plants or post-harvest crops are generally applied by crop growers each season.

For flowering plants, the compounds and compositions of the present invention may improve flower freshness of cut flowers. For flowering plants, the compounds and compositions of the present invention may increase flower duration of flowers that are not cut. For example, the compounds and compositions of the present invention may increase flower duration in pot plants and bedding plants, or in window boxes, grow-bags, gardens, parks, etc.

In order to apply the compound or composition of the invention to the plant or environs of the plant, the compound or composition may be used as a concentrate or more usually is formulated into a composition which includes an effective amount of the compound or composition of the present invention together with a suitable inert diluent, carrier material and/or surface active agent. Preferably the composition is in the form of an aqueous solution which may be prepared from the concentrate. By effective amount we mean that the composition (and/or its individual components) provides the effect of delaying abscission and/or improving post-harvest storability of crops, where required.

For application to plants and crops, the agent responsible for delaying abscission and/or improving post-harvest storability of crops is applied in a formulation that is preferably a substantially aqueous solution. The solution can be mixed on site in the spray tank or delivered and stored in aqueous solution, to ensure proper mixing and dilution, as appropriate.

The applied concentration of the agent responsible for delaying abscission and/or improving post-harvest storability of crops can vary widely depending on the water volume applied to plants as well as other factors such as plant age and size, and plant sensitivity to the agent. Typical rates of AN-related compounds would be 1-100 g/ha (preferably and in these trials, 1 g per hectare was applied), typical rates of acetaminophen or its derivatives would be 1-300 g/ha (preferably and in these trials, 3 g per hectare was applied). Typical rates of the agrochemically acceptable additive of the present invention would be 1-10 g/ha (preferably and in these trials, less than 3 g per hectare was applied). The rate of other components such as spreaders and stickers can be 50-200 ml per ha.

The rate and timing of application will depend on a number of factors known to those skilled in the art, such as the type of species etc. A second or further application(s) can be made as appropriate. The timings between each application may be in the region of 5 days or more. For maintenance of flower freshness, the timings between each application may be in the region of 3 days or more.

The present invention relates to a method of delaying the abscission of fruits, flowers, petals, seeds, leaves, stems or reducing pod shatter abscission and/or improving post-harvest storability of crops which comprises applying to the plants or to the locus thereof or to a harvested crop an effective controlling amount of the compound/compositions of the present invention.

The compounds and compositions of the present invention can be applied to the soil, plant, seed, or other area to be protected. Preferably the present invention is applied to the foliage of plants. The composition may be applied in the form of dusting powders, wettable powders, granules (slow or fast release), water dispersible granules, emulsion or suspension concentrates, liquid solutions, emulsions, seed dressings, or controlled release formulations such as microencapsulated granules or suspensions, soil drench, irrigation component, or preferably a foliar spray.

Dusting powders are formulated by mixing the active ingredient with one or more finely divided solid carriers and/or diluents, for example natural clays, kaolin, pyrophyllite, bentonite, alumina, montmorillonite, kieselguhr, chalk, diatomaceous earths, calcium phosphates, calcium and magnesium carbonates, sulfur, lime, flours, talc and other organic and inorganic solid carriers.

Granules are formed either by absorbing the active ingredient in a porous granular material for example pumice, attapulgite clays, fuller's earth, kieselguhr, diatomaceous earths, ground corn cobs, and the like, or on to hard core materials such as sands, silicates, mineral carbonates, sulfates, phosphates, or the like. Agents which are commonly used to aid in impregnation, binding or coating the solid carriers include aliphatic and aromatic petroleum solvents, alcohols, polyvinyl acetates, polyvinyl alcohols, ethers, ketones, esters, dextrins, sugars and vegetable oils, with the active ingredient. Other additives may also be included, such as emulsifying agents, wetting agents or dispersing agents.

Microencapsulated formulations (microcapsule suspensions CS) or other controlled release formulations may also be used, particularly for slow release over a period of time, and for seed treatment.

Alternatively and preferred the compositions may be in the form of liquid preparations to be used as dips, irrigation additives or sprays, which are generally aqueous dispersions or emulsions of the active ingredient in the presence of one or more known wetting agents, dispersing agents or emulsifying agents (surface active agents). The compositions which are to be used in the form of aqueous dispersions or emulsions are generally supplied in the form of an emulsifiable concentrate (EC) or a suspension concentrate (SC) containing a high proportion of the active ingredient or ingredients. An EC is a homogeneous liquid composition, usually containing the active ingredient dissolved in a substantially non-volatile organic solvent. An SC is a fine particle size dispersion of solid active ingredient in water. To apply the concentrates they are diluted in water and are usually applied by means of a spray to the area to be treated.

Suitable liquid solvents for ECs include methyl ketone, methyl isobutyl ketone, cyclohexanone, xylenes, toluene, chlorobenzene, paraffins, kerosene, white oil, alcohols (for example, butanol), methylnaphthalene, trimethylbenzene, trichloroethylene, N-methyl-2-pyrrolidone and tetrahydrofurfuryl alcohol (THFA).

These concentrates are often required to withstand storage for prolonged periods and after such storage, to be capable of dilution with water to form aqueous preparations which remain homogeneous for a sufficient time to enable them to be applied by conventional spray equipment. The concentrates may contain 1-85% by weight of the active ingredient or ingredients. When diluted to form aqueous preparations such preparations may contain varying amounts of the active ingredient depending upon the purpose for which they are to be used.

The composition may also be formulated as powders (dry seed treatment DS or water dispersible powder WS) or liquids (flowable concentrate FS, liquid seed treatment LS), or microcapsule suspensions CS for use in seed treatments. The formulations can be applied to the seed by standard techniques and through conventional seed treaters. In use the compositions are applied to the plants, to the locus of the plants, by any of the known means of applying fertiliser compositions, for example, by dusting, spraying, or incorporation of granules.

As indicated above, the compounds or compositions produced according to this present invention are usually applied to the foliage of plants but may also be applied to the soil or added to the irrigation water.

The compounds or compositions of the present invention can also be applied post-harvest to either whole plants with the root intact or directly to the harvested crop itself. The crop can include all types of fruit or vegetables and all types of agricultural and horticultural crops or produce that are kept for any length of time before marketing and/or consumption. In this case the composition may be applied to the whole plant or to the harvested crop by means of post-harvest sprays, dips or drenches.

In a particularly preferred embodiment, the one or more compounds of the invention are administered in combination optionally with one or more active agents. In such cases, the compounds of the invention may be administered consecutively, simultaneously or sequentially with each other or the one or more active agents. The major advantages of combining the compounds are that it may promote additive or possible synergistic effects through e.g. biochemical interactions. Beneficial combinations may be suggested by studying the activity of the test compounds. This procedure can also be used to determine the order of administration of the agents, i.e. before, simultaneously or after delivery.

It will be appreciated that the present invention may be applicable to all horticultural and agricultural species.

The present invention is particularly useful in relation to crops, either pre-harvest or post-harvest.

The present invention is useful in relation to fruit crops. The crops can include trees, bushes, shrubs and vines.

The present invention is useful in relation to vegetable crops.

The present invention is useful in relation to agricultural and horticultural crops including ornamentals.

The present invention is useful in relation to cereals and grasses and to pod-bearing, bean and oilseed crops.

The present invention can be used on pre-harvest plants and post-harvest crops such as:

Almond (Prunus dulcis), Apple (Malus domestica), Apricot (Prunus armeniaca), Avocado (Persea americana), Banana, Plantain (Musa spp.), Blackberries (Rubus spp), Blueberries (Vaccinium spp), Cacao or cocoa (Theobroma cacao), Cashew (Anacardium occidentale), Chemies (Prunus cerasus, P. avium), Chestnuts (Castanea spp.), Coconut (Cocos nucifera), Coffee (Coffea arabica, C. canephora), Cranberry (Vaccinium macrocarpon), Currants (Ribes spp), Date (Phoenix dactylifera), Fig (Ficus caricai), Gooseberry (Ribes grossularia; R. hirtellum), Grapefruit (Citrus paradisi), Grapes (Vitis vinifera, other Vitis spp), Guava (Psidium guajava & related spp), Hazelnut or filbert (Corylus avellana), Juneberry (Amelanchier alnifolia), Kiwifruit (Actinidia deliciosa), Kumquat (Fortunella spp), Lemon (Citrus limon), Lime (Citrus aurantifolia), Loquat (Eriobotrya japonica), Macadamia (Macadamia integrifolia), Mango (Mangifera indica), Mayhaw (Crataegus spp.), Nashi (Pyrus pyrifolia), Oil Palm (Elaeis guineensis), Olive (Olea europaea), Orange (Citrus sinensis), Papaya (Carica papaya), Peach (Prunus persica), Pears (Pyrus communis, P. pyrifolia), Pecan (Carya illinoensis), Pineapple (Ananas comosus), Pistachio (Pistacia vera), Plums (Prunus domestica, P. salicina), Pomegranate (Punica granatum), Quince (Cyclonia oblonga), Raspberries (Rubus idaeus, R. occidentalis), Strawberry (Fragaria X ananassa), Tangerine (Citrus reticulata), Walnut (Juglans regia)

The present invention can be used on pre-harvest plants and post-harvest crops such as:

Chrysanthemum, Rhododendron including Azalea species (e.g. Azaleastrum), Kalanchoe, Bulb crops, Crocus, Tulip, Narcissus, Hyacinth, Poinsettia, Roses and Poppy.

The present invention can also be used on pre-harvest plants and post-harvest crops such as:

Tomatoes, squash, pumpkin, beans, broccoli, green beans, asparagus, peas, corn, carrots, spinach, cauliflower, lima beans, broad beans, french beans, runner beans, navy beans, kidney beans, lentils, cabbage, onions, courgettes, aubergines, sweet basil, leeks, artichokes, lettuce, cassaya leaves, tomatoes, cucumbers and gherkins, marrows, gourds, squashes, chillies and peppers, green onions, dry onions, red onions, shallots, garlic, chives, other alliaceous vegetables, okra, mushrooms, watermelons, cantaloupe melons, other melons, bamboo shoots, beets, chards, capers, cardoons, celery, chervil, cress, fennel, horseradish, marjoram, oyster plant; parsley, parsnips, potato, radish, rhubarb, rutabaga, savory, scorzonera, sorrel, sprouts, swede, turnip, watercress and other vegetables.

The present invention can also be used on pre-harvest plants and post-harvest crops such as:

Maize, wheat, rye, oat, triticale, rice, barley, sorghum, millet, buckwheat, fonio, quinoa, spelt, other cereal crops, soybean, peanut, cotton, oilseed rape, sugar cane, bamboo, sesame, jute, canola, coconut, manihot, sunflower, tobacco, ground nuts, peanuts, oil palm, hemp, flax, lucerne, alfalfa tea, perennial grass.

The flower longevity and abscission point may be correlated.

The following mixtures of the compound or composition of the present invention are particularly mentioned:

1. The addition of Anthranilic Acid (AN).
2. The addition of Acetaminophen (AC) to Anthranilic Acid (AN).
3. The addition of an additive (ADD) to Anthranilic Acid.
4. The addition of ADD to AN+AC.
5. The addition of ADD to NAA/other auxins, or auxin mixes (eg NAA/BA).
6. The addition of ADD to AC+AN/NAA/other auxins, or auxin mixes (eg NAA/BA).
7. The addition to AC to NAA/other auxins or auxin mixes.

These and other combinations in accordance with the present invention may give rise to additive or synergistic effects.

The additive may be one set out as classes (a) to (f) above.

When the additive is selected from class (a) it is preferably one or more of glucose, sucrose, fructose or glycerol.

When the additive is selected from class (b) it is preferably one or more of citric or succinic acid.

When the additive is selected from class (c) it is preferably one or more of thiamine, riboflavin, pyridoxine, nicotinamide, folic acid, ascorbic acid, biotin or vitamin B12.

When the additive is selected from class (d) it is preferably adenine, thymidine, cytosine or uracil.

When the additive is selected from class (e) it is preferably a corn oil.

When the additive is selected from an amino acid is it preferably one of more of glycine, alanine, valine, leucine, threonine, cysteine, methionine, glutamine, asparagine or lysine.

The following Examples further illustrate, but do not limit, the invention.

### EXPERIMENTAL RESULTS

The TAMPF2 formulation example in these experiments included AN+AC+ADD, applied at the rate of 1 litre per hectare. ADD=at least one from class (f) each at <3 g/l, plus at least one from class (c).

The Lugol Test is a measure of the starch index and indicates the level of maturity of the fruit, measured on a scale of (0-9) with higher numbers equaling a higher level of maturity (0=least mature; 9=most mature). The results of this test show whether undesirable premature ripening has taken place.

A. Abscission Delay

PEAR, cv CONFERENCE. Sprayed (single application) three weeks before anticipated harvest. NAA (applied as formulation Fixor) was applied at its recommended rate. Both TAMPF2 and NAA were applied in 1000 litres water per hectare.

| | No. of | No. of | Mean | | | |
|---|---|---|---|---|---|---|
| | Fruits | Fruits | Value | Lugol | Lugol | |
| | under | under | Fallen | Test | Test | Mean Value |
| | Trees on | Trees on | Fruits | (0-9) | (0-9) | Lugol Test |
| Treatment | 22/9 | 14/10 | (22/9) | 9/09 | 16/9 | (0-9) (16/9) |
| Untreated | | | | | | |
| Rep 1 | 11.0 | 86.0 | 17.5 | 6.7 | 6.5 | 8.21 |
| Rep 2 | 13.0 | 92.0 | | 6.9 | 8.9 | |
| Rep 3 | 32.0 | 207.0 | | 8.3 | 9.4 | |
| Rep 4 | 14.0 | 70.0 | | 7.7 | 8.2 | |
| NAA | | | | | | |
| (Fixor) | | | | | | |
| Rep 1 | 6.0 | 63.0 | 7.0 | 7.8 | 8.5 | 8.62 |
| Rep 2 | 2.0 | 1.8 | | 7.0 | 7.4 | |
| Rep 3 | 12.0 | 113.0 | | 9.3 | 9.5 | |
| Rep 4 | 8.0 | 63.0 | | 7.5 | 9.1 | |
| TAMPF2 | | | | | | |
| Rep 1 | 3.0 | 55.0 | 8.5 | 7.8 | 6.0 | 7.38 |
| Rep 2 | 6.0 | 56.0 | | 6.8 | 7.0 | |
| Rep 3 | 24.0 | 211.0 | | 9.1 | 8.4 | |
| Rep 4 | 1.0 | 66.0 | | 6.8 | 8.2 | |

The results show effective abscission delay from the TAMPF2 formulation. The TAMPF2 formulation shows a lower number of fallen fruits, and no premature ripening.

The TAMPF2 formulation used in this experiment proved to be very safe, with no phytotoxicity or any damage whatsoever, and with no detrimental effect on maturity i.e. no premature ripening as is common with e.g. NAA (see Lugol test results).

B. Storage Ability

PEAR, cv CONFERENCE. Sprayed (two applications) two weeks+one week before anticipated harvest. NAA (applied as formulation Fixor) was applied at its recommended rates. Both TAMPF2 and NAA were applied in 1000 litres water per hectare. All fruits were harvested on the same date.

| | Lugol Test | Lugol Test | Mean | Fruit | Mean Fruit |
|---|---|---|---|---|---|
| | (0-9) High | (0-9) High | Value | Quality 4 wks | Quality 4 wks |
| | number = | number = | Lugol | after Storage | after Storage |
| | earlier | earlier | Test | Fruit Firmness | Fruit Firmness |
| | maturity | maturity | (0-9) | (kg/0.5 cm²) | (kg/0.5 cm²) |
| Treatment | 9/09 | 16/9 | (16/9) | 16/10 | 16/10 |
| Untreated | | | | | |
| Rep 1 | 6.7 | 6.5 | 8.21 | 5.7 | 5.28 |
| Rep 2 | 6.9 | 8.9 | | 4.8 | |
| Rep 3 | 8.3 | 9.4 | | 5.2 | |
| Rep 4 | 7.7 | 8.2 | | 5.5 | |
| NAA | | | | | |
| (Fixor) | | | | | |
| Rep 1 | 8.9 | 9.1 | 8.31 | 4.9 | 5.19 |
| Rep 2 | 8.5 | 7.4 | | 5.1 | |
| Rep 3 | 8.6 | 9.3 | | 5.0 | |
| Rep 4 | 8.7 | 7.5 | | 5.7 | |
| TAMPF2 | | | | | |
| Rep 1 | 8.0 | 6.9 | 8.11 | 5.4 | 5.87 |
| Rep 2 | 8.6 | 7.3 | | 6.0 | |
| Rep 3 | 9.3 | 9.3 | | 6.2 | |
| Rep 4 | 7.6 | 9.1 | | 6.0 | |

The results show an improvement in storability from the TAMPF2 formulation. The Lugol test showed no effect on ripening, but fruit firmness (a measure of fruit quality and storage ability) was significantly improved, in this experiment by over 11%, which equates to a longer period of storability. Fruit firmness was measured on fruits harvested on the same date, and with all skins removed, to demonstrate that this was not an effect caused by delayed fruit maturity. NAA application did not show any improvements in storage ability.

C. Abscission Delay

### 1. PAPAVER Nudicaule (Iceland Poppy)

Cultural Details: plants were sown in a commercial seed compost and then “pricked out” into a professional grade all-purpose compost in 9 cm pots set out in a randomized block design. Normal daylight plus supplementary lighting to provide a minimum of 10 hours per day. Heating was given to a minimum of 60 degrees Fahrenheit during the day and 40 degrees at night.

Sowing Date: 18 Nov. 2009, in plugs. Application Date: 5 Jan. 2010.

Measurement Date: 12 Jan. 2010 at daily intervals.

A number of auxins or auxin precursors were applied, each at 10⁻² Molar solutions. Addition of acetaminophen (AC) was at 3 g per hectare equivalent in each test. Measurement: Delay of first petal fall for each treatment (in days).

| | Total | | % |
|---|---|---|---|
| | Duration of | % Improvement | Improve- |
| | Flowering | from addition of AC | ment |
| | (to petal | to auxin/precursor. | over |
| Treatment | fall) | ie (B)/(A) × 100 | control |
| Control (C) | 9.8 | — | +0.0 |
| AC | 9.9 | — | +0.0 |
| Anthranilic acid (A) | 12.2 | — | +24.5 |
| Anthranilic acid + AC | 14.1 | +15.6 | +43.9 |
| (B) | | | |
| Anthranilic acid methyl | 12.1 | — | +23.5 |
| ester (A) | | | |
| Anthranilic acid methyl | 13.9 | +14.9 | +41.8 |
| ester + AC (B) | | | |
| Acetamide (A) | 11.7 | — | +19.4 |
| Acetamide + AC (B) | 13.8 | +17.9 | +40.8 |
| Anthranilic Acid | 11.7 | — | +19.4 |
| monosodium salt (A) | | | |
| Anthranilic Acid | 14.0 | +19.7 | +42.9 |
| monosodium salt + AC | | | |
| (B) | | | |
| Indole-3-acetamide (A) | 11.9 | — | +21.4 |
| Indole-3-acetamide + | 13.7 | +15.1 | +39.8 |
| AC (B) | | | |
| 3-Hydroxyanthranilic | 11.6 | — | +18.4 |
| acid (A) | | | |
| 3-Hydroxyanthranilic | 14.1 | +21.6 | +43.9 |
| acid + AC (B) | | | |
| Tryptamine (A) | 11.7 | — | +19.4 |
| Tryptamine + AC (B) | 13.7 | +17.1 | +39.8 |
| L-Tryptophan (A) | 10.8 | — | +10.2 |
| L-Tryptophan + AC | 12.6 | +16.7 | +28.6 |
| (B) | | | |

Petal abscission is delayed by the application of an auxin/auxin precursor. Petal abscission is further delayed by the application of an auxin/auxin precursor plus acetaminophen. Thus, flower longevity is benefited.

### 2. PAPAVER Nudicaule (Iceland Poppy)

To demonstrate the benefits to flower longevity (delay of petal abscission) from applying anthranilic acid (1 gram per litre solution, applied in 250 litres water per hectare, =1 g active total per hectare) or anthranilic acid plus acetaminophen (1 gram per litre and 3 gram per litre solution respectively, applied in 250 litres water per hectare, =1 g and 3 g actives total respectively per hectare). To each were added certain agrochemically acceptable additives, as examples. Rates per ha are provided in the Table below.

Cultural Details: plants were sown in a commercial seed compost and then “pricked out” into a professional grade all-purpose compost in 9 cm pots set out in a randomized block design. Normal daylight plus supplementary lighting to provide a minimum of 10 hours per day. Heating was given to a minimum of 60 degrees Fahrenheit during the day and 40 degrees at night.

Sowing Date: 19 Dec. 2009, in plugs. Application Date: 7 Feb. 2010.

Measurement Date: 12 Feb. 2010, at daily intervals.

Measurement: Delay of first petal-fall for each treatment (in days).

| | Total | % | |
|---|---|---|---|
| | duration of | Improve- | % Improvement |
| | flowering | ment | over appropriate |
| | (to petal | over | control (ie AN or |
| Treatment | fall) | untreated | AN + AC) |
| Untreated | 8.8 | — | — |
| Acetaminophen (AC) | 8.8 | — | — |
| Anthranilic acid (AN) | 10.9 | +23.9 | +0.0 |
| AN + AC | 13.1 | +48.9 | +0.0 |
| AN + Agrochemically | 11.9 | +35.2 | +9.2 |
| acceptable additive | | | |
| (AAA): Glucose 150 g | | | |
| (category (a) example) | | | |
| AN + AC + | 14.1 | +60.2 | +7.6 |
| Agrochemically | | | |
| acceptable additive | | | |
| (AAA): Glucose 150 g | | | |
| (category (a) example) | | | |
| AN + Agrochemically | 12.0 | +36.4 | +10.1 |
| acceptable additive | | | |
| (AAA): Succinic acid | | | |
| 50 g (category (b) | | | |
| example) | | | |
| AN + AC + | 14.3 | +62.5 | +9.2 |
| Agrochemically | | | |
| acceptable additive | | | |
| (AAA): Succinic acid | | | |
| 50 g (category (b) | | | |
| example) | | | |
| AN + Agrochemically | 12.1 | +37.5 | +11.0 |
| acceptable additive | | | |
| (AAA): Ascorbic acid | | | |
| 50 g (category (c) | | | |
| example) | | | |
| AN + AC + | 13.9 | +58.0 | +6.1 |
| Agrochemically | | | |
| acceptable additive | | | |
| (AAA): Ascorbic acid | | | |
| 50 g (category (c) | | | |
| example) | | | |
| AN + Agrochemically | 12.0 | +36.4 | +10.1 |
| acceptable additive | | | |
| (AAA): Corn oil 500 g | | | |
| (category (e) example) | | | |
| AN + AC + | 14.2 | +61.4 | +8.4 |
| Agrochemically | | | |
| acceptable additive | | | |
| (AAA): Corn oil 500 g | | | |
| (category (e) example) | | | |
| AN + Agrochemically | 11.8 | +34.1 | +8.3 |
| acceptable additive | | | |
| (AAA): L-arginine 30 g | | | |
| (category (f) example) | | | |
| AN + AC + | 14.0 | +59.1 | +6.9 |
| Agrochemically | | | |
| acceptable additive | | | |
| (AAA): L-arginine 30 g | | | |
| (category (f) example) | | | |

### Shelf-Life

### 1. RADISH—cv F1 Apache

To demonstrate the benefits to post-harvest shelf-life from adding an auxin/auxin precursor or auxin/auxin precursor plus acetaminophen.

Cultural Details: plants were sown in a commercial seed compost and then “pricked out” into a professional grade all-purpose compost in 9 cm pots set out in a randomized block design. Normal daylight plus supplementary lighting to provide a minimum of 10 hours per day. Heating was given to a minimum of 60 degrees Fahrenheit during the day and 40 degrees at night.

Sowing Date: 3 Nov. 2009, in plugs. Application Dates: 28 Dec. 2009 fb 4 Jan. 2010. Harvest Date: 11 Jan. 2010. Measurement: Shelf-Life (days after harvest) at ambient temperature.

A number of auxins or auxin precursors were compared, each at 10⁻² Molar solutions. Addition of acetaminophen (AC) was at 3 g per hectare equivalent in each test.

| | Shelf-Life | % Improvement | % |
|---|---|---|---|
| | Duration | from addition of | Improve- |
| | (days | AC to auxin/ | ment |
| | after | precursor. ie | over |
| Treatment | harvest) | (B)/(A) × 100 | control |
| Control (C) | 6.3 | — | +0.0 |
| AC | 6.2 | — | +0.0 |
| Anthranilic acid (A) | 8.6 | — | +36.5 |
| Anthranilic acid + AC (B) | 9.9 | +15.1 | +57.1 |
| Anthranilic acid methyl ester | 8.7 | — | +38.1 |
| (A) | | | |
| Anthranilic acid methyl | 9.9 | +13.8 | +57.1 |
| ester + AC (B) | | | |
| Acetamide (A) | 8.7 | — | +38.1 |
| Acetamide + AC (B) | 9.8 | +12.6 | +55.6 |
| Anthranilic | 7.7 | — | +22.2 |
| Acid monosodium salt (A) | | | |
| Anthranilic Acid | 8.9 | +15.6 | +41.3 |
| monosodium salt + AC (B) | | | |
| Indole-3-acetamide (A) | 6.5 | — | +3.2 |
| Indole-3-acetamide + AC (B) | 7.0 | +7.7 | +11.1 |
| 3-Hydroxyanthranilic acid | 7.3 | — | +15.9 |
| (A) | | | |
| 3-Hydroxyanthranilic acid + | 8.8 | +20.5 | +39.7 |
| AC (B) | | | |
| Tryptamine (A) | 7.0 | — | +11.1 |
| Tryptamine + AC (B) | 7.5 | +7.1 | +19.0 |
| L-Tryptophan (A) | 6.5 | — | +3.2 |
| L-Tryptophan + AC (B) | 7.2 | +10.8 | +14.3 |

### 2. RADISH—cv F1 Apache

To demonstrate the benefits to post-harvest shelf life from applying anthranilic acid (1 gram per litre solution, applied in 250 litres water per hectare, =1 g active total per hectare) or anthranilic acid plus acetaminophen (1 gram per litre and 3 gram per litre solution respectively, applied in 250 litres water per hectare, =1 g and 3 g actives total respectively per hectare). To each were added certain agrochemically acceptable additives, as examples. Rates per ha are provided in the Table below.

Cultural Details: plants were sown in a commercial seed compost and then “pricked out” into a professional grade all-purpose compost in 9 cm pots set out in a randomized block design. Normal daylight plus supplementary lighting to provide a minimum of 10 hours per day. Heating was given to a minimum of 60 degrees Fahrenheit during the day and 40 degrees at night.

Sowing Date: 28 Jan. 2010, in plugs. Application Date: 5 Mar. 2010.

Measurement Date: 13 Mar. 2010.
Measurement: Shelf-life duration (days).

| | Shelf-life | % | |
|---|---|---|---|
| | Duration | Improve- | % Improvement |
| | (days | ment | over appropriate |
| | after | over | control (ie AN or |
| Treatment | harvest) | untreated | AN + AC) |
| Untreated | 5.9 | — | — |
| Acetaminophen (AC) | 5.8 | — | — |
| Anthranilic acid (AN) | 8.5 | +44.1 | — |
| AN + AC | 9.7 | +64.4 | — |
| AN + Agrochemically | 9.3 | +57.6 | +9.4 |
| acceptable additive | | | |
| (AAA): Glucose 150 g | | | |
| (category (a) example) | | | |
| AN + AC + | 10.6 | +79.7 | +9.3 |
| Agrochemically | | | |
| acceptable additive | | | |
| (AAA): Glucose 150 g | | | |
| (category (a) example) | | | |
| AN + Agrochemically | 9.4 | +59.3 | +10.6 |
| acceptable additive | | | |
| (AAA): Succinic acid | | | |
| 50 g (category (b) | | | |
| example) | | | |
| AN + AC + | 10.7 | +81.3 | +10.3 |
| Agrochemically | | | |
| acceptable additive | | | |
| (AAA): Succinic acid | | | |
| 50 g (category (b) | | | |
| example) | | | |
| AN + Agrochemically | 9.3 | +57.6 | +9.4 |
| acceptable additive | | | |
| (AAA): Ascorbic acid | | | |
| 50 g (category (c) | | | |
| example) | | | |
| AN + AC + | 10.6 | +79.7 | +9.3 |
| Agrochemically | | | |
| acceptable additive | | | |
| (AAA): Ascorbic acid | | | |
| 50 g (category (c) | | | |
| example) | | | |
| AN + Agrochemically | 9.5 | +61.0 | +11.8 |
| acceptable additive | | | |
| (AAA): Corn oil 500 g | | | |
| (category (e) example | | | |
| AN + AC + | 10.9 | +84.7 | +12.4 |
| Agrochemically | | | |
| acceptable additive | | | |
| (AAA): Corn oil 500 g | | | |
| (category (e) example | | | |
| AN + Agrochemically | 9.4 | +59.3 | +10.6 |
| acceptable additive | | | |
| (AAA): L-arginine 30 g | | | |
| (category (f) example) | | | |
| AN + AC + | 10.7 | +81.4 | +10.3 |
| Agrochemically | | | |
| acceptable additive | | | |
| (AAA): L-arginine 30 g | | | |
| (category (f) example) | | | |

All publications mentioned in the above specification are herein incorporated by reference. Various modifications and variations of the described methods and systems of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the field are intended to be within the scope of the following claims.

1.-76. (canceled)

77. A composition comprising a compound selected from an auxin, an auxin precursor, an auxin metabolite or a derivative of said auxin, auxin precursor or auxin metabolite or a mixture thereof and optionally acetaminophen or a derivative thereof for improving post-harvest storability of crops.

78. A composition comprising a compound selected from an auxin, an auxin precursor, an auxin metabolite or a derivative of said auxin, auxin precursor or auxin metabolite or a mixture thereof or a mixture thereof, acetaminophen or a derivative thereof and optionally an agrochemically acceptable additive for improving post-harvest storability of crops;
wherein the agrochemically acceptable additive preferably comprises at least one compound selected from:
a) glucose, hydrolysed starch, sucrose, fructose, glycerol, glyceraldehydes, erythrose, ribulose, xylulose or arabinose, monosaccharides including aldoses such as D-Ribose, D-Xylose, L-Arabinose, D-Glucose, D-Mannose and D-Galactose; ketoses such as D-Ribulose and D-Fructose; deoxyaldoses such as 2-Deoxy-D-ribose, L-Fuccose; acetylated amino sugars such as N-Acteyl-D-glucosamine and N-Acetyl-D-galactosamine; acidic monosaccharides such as D-Glucuronic acid, L-Iduronic acid and N-Acetylneuraminic acid, Sugar alcohols such as D-Sorbitol and D-Mannitol, disaccharides including maltose, lactose and sucrose, or an ester or glycoside or metabolic equivalent of such a carbohydrate;
b) an organic acid of the Krebs tricarboxylic acid cycle or a metabolic precursor thereof;
c) a vitamin or coenzyme, or a precursor thereof;
d) a purine or pyrimidine nucleoside, nucleotide or metabolic precursor thereof; e) a naturally occurring fat or oil; or
f) an amino acid; and more preferably
comprises at least one compound selected from c) a vitamin or coenzyme, or a precursor thereof; d) a purine or pyrimidine nucleoside, nucleotide or metabolic precursor thereof; or f) an amino acid.

79. The composition according to claim 77 wherein the auxin is an indolic auxin or a phenolic auxin.

80. The composition according to claim 77 wherein the precursor is chorismate, anthranilic acid, phosphoribosyl anthraniliate, 1-(O-carboxyphenulamino)-1-deoxyribulose-5-phosphate, indole-3-glycerol-phosphate, indole, indole-3-acetic acid, tryptophan, tryptamine, N-hydroxy tryptamine, indole-3-acetaldoxime, 1-aci-nitro-2-indolylethane, indolic glucosinate, indole-3-acetonitrile (IAN), indole-3-acetaldehyde, indole-3-lactic acid, indole-3-pyruvic acid, or indole-3-ethanol; preferably anthranilic acid.

81. The composition according to claim 77 wherein the auxin is a natural auxin selected from indole-3-acetic acid (IAA), 4-chloro-indole-3-acetic acid (4-Cl-IAA), phenylacetic acid (PAA), indole-3-butyric acid (IBA), indole-3-acetyl-1-O-β-D-glucose (IAAglc); or a synthetic auxin selected from 1-naphthaleneacetic acid (NAA), 2,4-dichlorophenoxyacetic acid (2,4-D), 2-methoxy-3,6-dichlorobenzoic acid (dicamba), 4-amino-3,5,6-trichloropicolinic acid (tordon), 2,4,5-trichlorophenoxyacetic acid (2,4,5-T), 2,3,6-trichlorobenzoic acid, 4-chloro-2 methyl phenoxy acetic acid (MCPA) or N,N-dimethylethylthiocarbamate.

82. Anthranilic acid or a derivative thereof for improving post-harvest storability of crops.

83. A composition comprising a compound selected from an auxin, an auxin precursor, an auxin metabolite or a derivative of said auxin, auxin precursor or auxin metabolite or a mixture thereof and an agrochemically acceptable additive for improving post-harvest storability of crops.

84. A composition for improving post-harvest storability of crops comprising a or a mixture thereof or a composition of claim 77 in combination with a further abscission control or reduction agent;
wherein the further abscission control or reduction agent is preferably selected from 1-naphthaleneacetic acid (NAA), NAAm, 2,4-D, 2,4,5-T, 2,4,5-TP, amino-oxyacetic acid, aminoethoxyvinylglycine (AVG) and daminozide.

85. A method of improving post-harvest storability of crops comprising applying the composition of claim 77 to a plant or its environs; to a crop after harvest; to a harvested crop; or to a whole plant post-harvest; wherein
the crop is preferably a fruit, vegetable, cereal, grass, pod-bearing, bean or oilseed crop.

86. The method of claim 85 wherein crop storability is prolonged, quality of post-harvest crops is improved, onset of post-harvest diseases is delayed, and/or senescence is delayed.

87. A composition comprising a compound selected from an auxin, an auxin precursor, an auxin metabolite or a derivative of said auxin, auxin precursor or auxin metabolite or a mixture thereof and acetaminophen or a derivative thereof for delaying abscission in plants.

88. A composition comprising a compound selected from an auxin, an auxin precursor, an auxin metabolite or a derivative of said auxin, auxin precursor or auxin metabolite or a mixture thereof or a mixture thereof, acetaminophen or a derivative thereof and an agrochemically acceptable additive for delaying abscission in plants;
wherein the agrochemically acceptable additive preferably comprises at least one compound selected from
a) glucose, hydrolysed starch, sucrose, fructose, glycerol, glyceraldehydes, erythrose, ribulose, xylulose or arabinose, monosaccharides including aldoses such as D-Ribose, D-Xylose, L-Arabinose, D-Glucose, D-Mannose and D-Galactose; ketoses such as D-Ribulose and D-Fructose; deoxyaldoses such as 2-Deoxy-D-ribose, L-Fuccose; acetylated amino sugars such as N-Acteyl-D-glucosamine and N-Acetyl-D-galactosamine; acidic monosaccharides such as D-Glucuronic acid, L-Iduronic acid and N-Acetylneuraminic acid, Sugar alcohols such as D-Sorbitol and D-Mannitol, disaccharides including maltose, lactose and sucrose, or an ester or glycoside or metabolic equivalent of such a carbohydrate;
b) an organic acid of the Krebs tricarboxylic acid cycle or a metabolic precursor thereof;
c) a vitamin or coenzyme, or a precursor thereof;
d) a purine or pyrimidine nucleoside, nucleotide or metabolic precursor thereof;
e) a naturally occurring fat or oil; or
f) an amino acid; and more preferably
comprises at least one compound selected from c) a vitamin or coenzyme, or a precursor thereof; d) a purine or pyrimidine nucleoside, nucleotide or metabolic precursor thereof; or f) an amino acid.

89. The composition according to claim 87 wherein the auxin is an indolic auxin or a phenolic auxin.

90. The composition according to claim 87 wherein the precursor is chorismate, anthranilic acid, phosphoribosyl anthraniliate, 1-(O-carboxyphenulamino)-1-deoxyribulose-5-phosphate, indole-3-glycerol-phosphate, indole, indole-3-acetic acid, tryptophan, tryptamine, N-hydroxy tryptamine, indole-3-acetaldoxime, 1-aci-nitro-2-indolylethane, indolic glucosinate, indole-3-acetonitrile (IAN), indole-3-acetaldehyde, indole-3-lactic acid, indole-3-pyruvic acid, or indole-3-ethanol; preferably anthranilic acid.

91. The composition according to claim 87 wherein the auxin is a natural auxin selected from indole-3-acetic acid (IAA), 4-chloro-indole-3-acetic acid (4-Cl-IAA), phenylacetic acid (PAA), indole-3-butyric acid (IBA), indole-3-acetyl-1-O-β-D-glucose (IAAglc); or a synthetic auxin selected from 1-naphthaleneacetic acid (NAA), 2,4-dichlorophenoxyacetic acid (2,4-D), 2-methoxy-3,6-dichlorobenzoic acid (dicamba), 4-amino-3,5,6-trichloropicolinic acid (tordon), 2,4,5-trichlorophenoxyacetic acid (2,4,5-T), 2,3,6-trichlorobenzoic acid, 4-chloro-2 methyl phenoxy acetic acid (MCPA) or N,N-dimethylethylthiocarbamate.

92. Anthranilic acid or a derivative thereof for delaying abscission in plants.

93. A composition comprising a compound selected from an auxin, an auxin precursor, an auxin metabolite or a derivative of said auxin, auxin precursor or auxin metabolite or a mixture thereof and an agrochemically acceptable additive for delaying abscission in plants.

94. A composition for delaying abscission in plants comprising a composition of claim 87 in combination with a further abscission control or reduction agent; wherein the further abscission control or reduction agent is preferably selected from 1-naphthaleneacetic acid (NAA), NAAm, 2,4-D, 2,4,5-T, 2,4,5-TP, amino-oxyacetic acid, aminoethoxyvinylglycine (AVG) and daminozide.

95. A method of delaying abscission in plants comprising applying the composition of claim 87 to a plant or its environs;
wherein the plant is preferably a fruit, vegetable, cereal, grass, pod-bearing, bean or oilseed crop.

96. The method of claim 95 wherein fruit drop is delayed,
wherein seed loss is delayed, or
wherein pod shatter is reduced.
